(19) United States
(12) Patent Application Publication
(10) Pub. No.: US 2025/0037826 A1
(43) Pub. Date: Jan. 30, 2025

(54) PREDICTIVE MODELING FOR HEALTHCARE COSTS

(71) Applicant: Adding Value to the Healthcare Equation, Inc., Silver Spring, MD (US)

(72) Inventors: Thomas J. DAWSON, III, Silver Spring, MD (US); Muhammad Farhan Ullah BABAR, Peshawar (PK)

(73) Assignee: Adding Value to the Healthcare Equation, Inc., Silver Spring, MD (US)

(21) Appl. No.: 18/784,737

(22) Filed: Jul. 25, 2024

Related U.S. Application Data
(60) Provisional application No. 63/515,545, filed on Jul. 25, 2023.

(51) Int. Cl. G16H 20/00; G06Q 30/0201; G16H 10/60; G16H 15/00

(52) U.S. Cl. CPC G16H 20/00; G06Q 30/0206; G16H 10/60; G16H 15/00

(57) ABSTRACT

Techniques may include collecting a personal profile of a user. The techniques may include receiving information identifying an event location for an event associated with the user. The techniques may include receiving at least one remediation profile for remediation of the user, where each remediation profile may include one or more of a category of remediation, a remedial location designation, a preference type, a service length, a number of annual units, or an uncovered equivalent. Moreover, the techniques may include for each remediation profile: identifying a medical care preference system associated with the remediation profile from a plurality of data systems. The personal profile, the event location, and the remediation profile can be provided as input to the medical care preference system and a projection can be output for each remediation profile.

### CROSS-REFERENCES TO OTHER APPLICATIONS

This application claims priority to U.S. Provisional Application No. 63/515,545, for “PREDICTIVE MODELING FOR HEALTHCARE COSTS” filed on Jul. 25, 2023, which is herein incorporated by reference in its entirety for all purposes.

### BACKGROUND

Projecting the cost or remediation for an event can be a time-consuming manual process. Producing a single projection can take months and such projections are not easily modified or updated. A system for automated projections for remediation in response to an event is desirable.

### BRIEF SUMMARY

In one general aspect, techniques may include collecting a personal profile of a user. Techniques may also include receiving information identifying an event location for a event associated with the user. The techniques may furthermore include receiving at least one remediation profile for remediation of the user, where each remediation profile may include one or more of a category of remediation, a remedial location designation, a modality type, a service length, a number of annual units, or an uncovered equivalent. The techniques may in addition include for each remediation profile: identifying, from a plurality of modality systems, a modality system associated with the remediation profile, each modality system having one or more of an equivalency system, a equipment system, a statistics system, a remedial substance system, or a service system; providing the personal profile, the event location, and the remediation profile as input to the modality system; generating, using the modality system, a projection output for each remediation profile based on the personal profile; and providing, for presentation at a user device, information associated with the projection. The techniques can include corresponding methods, computer systems, apparatus, and computer programs recorded on one or more computer storage devices, each configured to perform the actions of the techniques.

In one general aspect, techniques may include receiving information identifying a remedial unit relating to providing remediation to a user. Techniques may also include retrieving a first equivalency corresponding to the remedial unit from an equivalency system. The techniques may furthermore include retrieving a local weight from a localization system, the local weight corresponding to a administrative region where the user is provided remediation. The techniques may in addition include adjusting the first equivalency by the local weight to determine a weighted first equivalency. The techniques may moreover include retrieving a local enterprise equivalent corresponding to the remedial unit, the local enterprise equivalent retrieved from a commercial rate database, the local cost corresponding to the administrative region. The techniques may also include retrieving a first administrative remediation unit corresponding to the remedial unit from a first administrative remediation unit database corresponding to the administrative region. The techniques may furthermore include retrieving a second administrative remediation unit corresponding to the remedial unit from a second administrative remediation unit database corresponding to the administrative region. The techniques may include retrieving a third administrative remediation unit corresponding to the remedial unit from a third administrative remediation unit database corresponding to the administrative region. The techniques may in addition include summing, by the computing system, the weighted first equivalency, the local enterprise equivalent, the first administrative remediation unit, the second administrative remediation unit, and the third administrative remediation unit to produce an equivalency corresponding to the remedial unit in the administrative region.

In one general aspect, techniques may include presenting a graphical user interface. The techniques may also include receiving, by the computer system and via the graphical user interface, at least one remediation profile for remediation of a user, where each remediation profile may include one or more of a category of remediation, a charging of the techniques, a modality type, a service length, a number of annual units, or an uncovered equivalent. The techniques may furthermore include displaying, by the computer system and via the graphical user interface, an interactive map. The techniques may in addition include receiving, by the computer system and via the graphical user interface, a first input identifying a first administrative region of the interactive map. The techniques may moreover include, responsive to receiving the first input, calculating a modality projection based at least in part on the at least one remediation profile and the first administrative region. The techniques may also include displaying, by the computer system and via the graphical user interface, the modality projection for the remediation of the user in the first administrative region.

In one general aspect, techniques may include presenting a graphical user interface. Techniques may also include receiving a personal profile for a user. The techniques may furthermore include displaying, by the computer system and via the graphical user interface, an interactive map. The techniques may in addition include receiving, by the computer system and via the graphical user interface, a first input identifying a first administrative region of the interactive map. The techniques may moreover include, responsive to receiving the first input, calculating a personal projection for the user in the first administrative region based at least in part on the personal profile and the first administrative region. The techniques may also include displaying, by the computer system and via the graphical user interface, the personal projection for the user in the first administrative region.

In one general aspect, the disclosed techniques may include collecting, by a computer system, a personal profile of an user. Techniques may also include receiving information identifying a first event location for an event associated with the user. The techniques may furthermore include receiving, by the computer system, at least one remediation profile for remediation of the user, where each remediation profile may include one or more of a category of remediation, a remedial location designation, a remediation preference type, a service length, a number of annual units, or an uncovered equivalent. The techniques may in addition include for each remediation profile: identifying, from a plurality of data systems, a set of data systems associated with the remediation profile, each data system having one or more of an equivalency system, an equipment system, a statistics system, a remedial substance system, or a service system; providing the personal profile, the first event location, and the remediation profile as input to the set of data systems; generating, using the set of data systems, a projection as output for each remediation profile; and providing, for presentation at an user device, information associated with the projection for each remediation profile.

Implementations may include one or more of the following features. Techniques where providing information associated with the projection for each remediation profile may include, for each remediation profile: determining, by the computer system, a remediation assessment score; generating, by the computer system, a visual representation of the remediation assessment score; and providing, by the computer system, instructions that cause an user device to display the visual representation as the information associated with the projection. Techniques where receiving information identifying the first event location may include: providing, by the computer system, instructions to cause the user device to display a graphical user interface having an interactive map; and receiving, by the computer system, information identifying a first event location from the user device, where the information identifying the first event location is provided as input to the interactive map. Techniques where providing the information associated with the projection for each remediation profile may include: receiving, by the computer system, information identifying a second event location from the user device, where the information identifying the second event location is provided as input to the interactive map; and updating, by the computer system, the projection based at least in part on the second event location. Techniques where receiving the at least one remediation profile may include: providing, by the computer system, instructions to cause the user device to display a graphical user interface; receiving, by the computer system and from the user device, one or more categories of remediation for the at least one remediation profile; querying, by the computer system, the plurality of data systems to identify a plurality of matching remediation preference types that correspond to the one or more categories of remediation; and providing, by the computer system, instructions to cause the user device to display at least one of the plurality of matching remediation preference types on the graphical user interface. The techniques may include: receiving, by the computer system, information identifying one or more remediation preference types of the plurality of matching remediation preference types; and adding, by the computer system, the one or more of the plurality of matching remediation preference types to the at least one remediation profile.

In one general aspect, the techniques may be performed by a computer system may include one or more non-transitory memories. The computer system may also include one or more processors in communication with the one or more memories and configured to execute instructions stored in the one or more memories to performing the techniques. Implementations of any of the described techniques may include hardware, a method or process, or a non-transitory computer tangible medium.

A better understanding of the nature and advantages of embodiments of the present disclosure may be gained with reference to the following detailed description and the accompanying drawings.

### BRIEF DESCRIPTION OF THE DRAWINGS

FIG. 1 shows a simplified diagram of a system for generating a projection according to various embodiments.

FIG. 2 shows a simplified diagram of a care system according to various embodiments.

FIG. 3 shows a simplified diagram of an equivalency system according to at least one embodiment.

FIG. 4 shows a graphical user interface with a dashboard according to at least one embodiment.

FIG. 5 shows a graphical user interface with an interactive map according to at least one embodiment.

FIG. 6 shows a graphical user interface with a modality calculator according to at least one embodiment.

FIG. 7 shows a graphical user interface with an interactive map and modality calculator according to at least one embodiment.

FIG. 8 shows a graphical user interface with a life expectancy calculator according to at least one embodiment.

FIG. 9 shows a graphical user interface for entering a case profile according to at least one embodiment.

FIG. 10 shows a graphical user interface for entering residence information for a case profile according to at least one embodiment.

FIG. 11 shows a graphical user interface for entering information about an injury location for a case profile according to at least one embodiment.

FIG. 12 shows a graphical user interface for entering information about an injury diagnosis for a case profile according to at least one embodiment.

FIG. 13 shows a graphical user interface for entering information about a subject's disability status for a case profile according to at least one embodiment.

FIG. 14 shows a graphical user interface for entering information about a subject's employment status for a case profile according to at least one embodiment.

FIG. 15 shows a graphical user interface for entering custom values for a projection.

FIG. 16 is a graphical user interface for comparing reasonable totals by state according to at least one embodiment.

FIG. 17 shows a graphical user interface for adding collaborators to a remediation profile according to at least one embodiment.

FIG. 18 shows a graphical user interface that displays information about a collaboration team according to at least one embodiment.

FIG. 19 shows a graphical user interface that displays notes shared with a collaboration team according to at least one embodiment.

FIG. 20 shows a graphical user interface for drafting a note according to at least one embodiment.

FIG. 21 shows a graphical user interface for searching for search modalities according to at least one embodiment.

FIG. 22 shows a graphical user interface displaying a starred remediation preference list according to at least one embodiment.

FIG. 23 shows a graphical user interface for adding remediation preferences to a starred remediation preference list according to at least one embodiment.

FIG. 24 shows a graphical user interface for creating custom remediation preferences according to at least one embodiment.

FIG. 25 shows a graphical user interface for creating a new remediation preference according to at least one embodiment.

FIG. 26 shows a graphical user interface for displaying an assessment score according to at least one embodiment.

FIG. 27 shows a graphical user interface 2700 for contextual remediation preference suggestions according to at least one embodiment.

FIG. 28 is a process for determining a projection according to at least one embodiment.

FIG. 29 is a process for determining an equivalency according to at least one embodiment.

FIG. 30 is a process for determining modality projection using an interactive map according to at least one embodiment.

FIG. 31 is a process for determining a personal projection using an interactive map according to at least one embodiment.

FIG. 32 is a process for performing a search according to at least one embodiment.

FIG. 33 is a process for displaying a visual representation of a remediation assessment score according to at least one embodiment.

FIG. 34 is a process for suggesting remediation preferences according to at least one embodiment.

FIG. 35 shows a simplified diagram of a computer system according to at least one embodiment.

### DETAILED DESCRIPTION OF THE INVENTION

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiment. It is understood that various changes may be made in the function and arrangement of elements without departing from the spirit and scope as set forth in the appended claims.

In one example, a projection (e.g., a care projection) can be generated for an injured individual. This care projection can be the estimated cost of remediation in response to an event (e.g., a treatment for an injury), and the projection can be used to plan for future remediation units (e.g., medical costs). For instance, a projection could be used to determine damages in personal injury litigation. Care related to an event (e.g., an injury) can involve one or more methods of treatment called remediation (e.g., medical care) preferences. A remediation preference can include surgery, medication, inpatient treatment, outpatient treatment, equipment, occupational therapy, physical therapy, and any other treatment related to the care of an injury.

A remediation profile (e.g., a medical care preference profile) can include each remediation preference that is used in the treatment of a particular injury, and a projection can be generated for each remediation profile. The remediation profile can include information that can be used as input to a model for determining the projection. In addition, the model can use a location for the event (e.g., the medical event) that caused the user's injury, and/or where the user will be treated, to provide a contextualized projection for that user. Such contextualization can be helpful as laws and regulations regarding providing healthcare, including those that impact healthcare costs, may vary depending on the location.

The present disclosure provides several technical advantages over existing solutions that improve the functioning of systems for generating projections for a user. Such projections can be time consuming to produce and the projections can be tied to a particular administrative region (e.g., geographic location) and set of remediation preferences. The personal profile (e.g. personal profile), event locations, and remediation profiles are configurable so that each can be changed individually. Medical care preferences can be individually added to a projection without the user having to contextualize each remediation profile. The contextual information can be shared by the system across remediation profiles so that a user can add remediation profiles without having to reenter all of the contextual information (e.g., injury location, personal profile, etc.). Such sharing allows the user to enter additional remediation profiles with fewer graphical user interface pages than were required for the first remediation profile.

In another example, a system can calculate an equivalency (e.g., a relative value unit) for a remedial unit (e.g., a medical care unit). The remedial unit can be a service or procedure that is performed by a physician or another medical processional. Comparing the cost of medical procedures can be difficult without a point of comparison between the different procedures. An equivalency can address this problem by assigning a procedure a value relative to all medical services and procedures. The equivalency can then be converted to dollars using a conversion factor of dollars per equivalency.

Relative value units (e.g., remedial units) are meant to compensate medical professionals for the effort and expense required to provide that remedial unit. The disclosed equivalency system calculates a localized equivalency for a specific administrative location. For example, an equivalency can be calculated for a medical procedure (e.g., a remedial unit) that is performed in a specific zip code. The equivalency can be calculated for a remedial unit using a weighted sum of a Medicare component, a Medicaid component, a state veteran's administration component, enterprise equivalent (e.g., commercial cost) component, and population data. These components can vary by location and the equivalency system can pull local data for each component from databases maintained by the system.

The Medicare component of the equivalency may include three components: (1) a work equivalency representing the work involved in providing the remedial unit, (2) practice expense equivalencies representing the non-malpractice overhead expenses in providing the remedial unit, and (3) malpractice equivalencies representing the cost of malpractice insurance related to the remedial unit. Medicare is a national government health insurance program in the United States, but the compensation for remedial units varies by state. To reflect these variations, the unweighted Medicare component of the equivalency (e.g., the first equivalency) is multiplied by a Medicare Geographic Practice Cost Index (GPCI; e.g., a local weight) to determine the Medicare component.

In addition to the Medicare component, the values for the Medicaid component, the state veteran's administration component, the enterprise equivalent component, and the population data can be obtained from databases associated with the disclosed system. The equivalency can be calculated by summing the values for each component. In some embodiments, the one or more of the values can be multiplied by a weight and the weighted values can be summed to produce the equivalency for a given remedial unit.

The present disclosure provides several technical advantages over existing solutions that improve the functioning of systems for calculating equivalencies. Such projections are not easily modified. The present system is modular and allows a user to update portions of the projection without having to calculate an entirely new projection. The disclosed system can allow for a remediation profile (e.g, treatment plan) to be modified or updated by allowing the user to access and change the relevant portions of the remediation profile (e.g., care plan) without having to submit redundant information. Such a system can allow a user to update a remediation profile using fewer graphical user interfaces than would be used in previous systems. For instance, the user can use a menu to navigate to the relevant portions of the remediation profile without clicking through graphical user interfaces for the portions that do not need to be updated.

In another example, a system can present a graphical user interface that can be used to determine projections (e.g., medical care preference projections) across administrative regions (e.g., geographic regions). Medical care preference projections, and the cost for treatment, can vary significantly between regions. A user may wish to evaluate the cost of treatment in different locations for a number of reasons. For instance, an injured individual, or their caretakers, may wish to see which areas are most cost effective in order to budget for long term care. The disclosed system dynamically calculates the projection based on user input to an interactive map (e.g., a graphical user interface). A user can provide the input by clicking, highlighting, or hovering over a location in the map and the graphical user interface can display a projection for the selected region. The system described herein then dynamically calculates the remediation profile for each selected region rather than for all of the regions displayed on the map.

The present disclosure provides several technical advantages over existing solutions that improve the functioning of systems for determining projections. The system is dynamic because the projection for a region is determined as needed in response to a user input. Other systems may calculate the remediation profile for a specific region rather than allowing a user to modify the system as needed. In addition, calculations that are generated or recalculated based on the user input to a map can conserve computing resources. For example, calculating a projection for each zip code may involve over 41,000 individual projections. Instead, projections for only a subset of the total number of zip codes may be calculated based on the user's input and this can significantly reduce the amount of computing resources that are needed to create a projection for a particular event.

In another example, life expectancies are used to calculate the projections for a subject. An annual cost for an item or service may have to be projected for the lifetime of a subject in order to accurately project the costs associated with that subject's treatment. However, life expectancy tables can be confusing to use and comparing life expectancies across regions can be difficult with life expectancy tables which are expansive and can include projections for over 41,000 zip codes. A system can present a graphical user interface that can be used to create a personal profile for the individual. This personal profile can include the information used to project a life expectancy as well as the projected life expectancy. Such a system could be used to evaluate locations where the user might want to live or to allow a user to plan for retirement expenses using a reasonable life expectancy for the user.

The personal profile, with the life expectancy projection, can be used to generate a projection, an equivalency and/or any other projection in this disclosure. By calculating a life expectancy through a single user interface, the process of calculating a life expectancy can be simplified. Also, the user of the system can be presented with fewer user interfaces so that calculating a projection or equivalency can require the user to click through fewer user interfaces. In addition, an interactive map can be used to select administrative regions and the relevant information about that region can be used to generate a projection. Reducing the amount of input that a user may need to provide in order to interact with a user interface can offer a technical benefit by making the user interfaces more accessible to users with physical or mental disabilities. The disclosed projections may be about remediation related to a user's physical or mental disability, and enabling users with physical or mental limitations to create or modify projections can allow them to obtain projections that require fewer revisions because the projections (with the user's input) accurately reflect the user's needs.

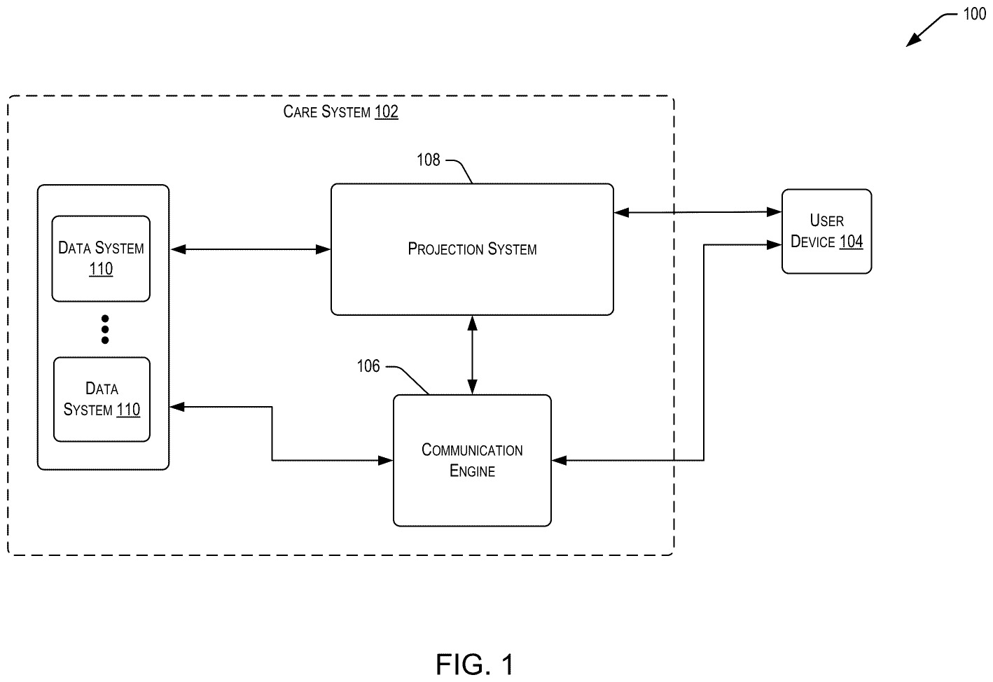

FIG. 1 shows a simplified diagram 100 of a system for generating a projection according to various embodiments. The diagram 100 includes a plurality of elements connected with directional arrows. The directional arrows not only indicate that the elements are connected, but also indicate the direction that data may flow with respect to the various elements. For example, data may flow between the following elements shown in diagram 100: a user device 104 and a communication engine 106. Any one of the systems and engines in this disclosure can be implemented as hardware, as software, or as a combination of software and hardware.

The care system 102 can collect, receive, or request a personal profile from user device 104. The personal profile can include information about the subject of the projection (e.g., a user; the person receiving care). A user controlling the user device 104 can provide a personal profile of an injured individual (e.g., the subject) to the care system 102. The personal profile can be used by the care system to produce a personal profile for the injured individual. The user may be an attorney preparing for personal injury litigation, a financial planner helping the injured individual plan for retirement costs (e.g., the costs related to care during retirement), an injured individual planning where to live during their convalescence, and/or any other individual who may want a remediation profile detailing the costs related to providing care to the subject during a fixed or variable duration. The personal profile can include the user's name, gender, citizenship status, ethnicity, marital status, residence, date of birth, injury location, any diagnoses (e.g., diagnostic codes), disability status, and employment status. The disability status can include information indicating whether the user is totally and permanently disabled, legally blind, has end stage renal diseases (ESRD), and if the user is part of any Medicaid waver programs. The employment status can include information indicating the user's employment status, primary insurance, household income, whether the user has a life remediation profile, and the life remediation profile date. In some embodiments, the personal profile can be retrieved from a database, and the personal profile can be associated with a user account.

The personal profile, and any other data, is transmitted throughout care system 102 in accordance with any suitable transmission protocol. Generally, the communication engine 106 is configured to manage the flow of such transmissions within the care system 102. Thus, the communication engine 106 receives indications of transmissions of projection data and tracks the origination locations of the transmissions, the destination locations of the transmissions, and any locations therebetween. Projection data can include a projection, or the information used to determine a projection.

The care system 102 may generate a care projection (e.g., a projection) in response to a request that contains one or more personal profiles and any additional user information that can be used to calculate a projection. The personal profile and request can be forwarded to the projection system 108 by the communication engine 106. The projection system 108 may retrieve projection data, in addition to the personal profile, from one or more data systems (e.g., medical care preference systems) 110. The projection data can include equivalencies, equipment (e.g., durable medical equipment) expenses, reasonable value for lost labor, remedial substances, the cost of providing care, and/or any other cost that may relate to providing care to the subject during a fixed or variable duration. This projection data can be collected from data systems 110, and, for example, the projection data can be retrieved from an equivalency system, an equipment (e.g., a durable medical equipment) system, a statistics system (e.g., a bureau of labor statistics system), a remedial substance system, or a service system respectively. The care system 102 may include one or more additional data systems (e.g., an inflation system or a housing cost system).

Equivalencies are a measure of the value of physician services, and equivalencies are used by Medicare to determine reimbursements to physicians. Equipment (e.g., durable medical equipment) expenses are expenses related to medical equipment that can be used repeatedly (e.g., the cost of purchasing equipment). Such equipment is generally equipment that a user uses to complete daily activities such as a walker, wheelchair, or oxygen tanks. In addition to the direct expenses involved in caring for an injured individual, the user may suffer harm if they are no longer able to work. A reasonable value for lost labor can be collected from a statistics system. Drug costs represent the cost of providing drugs or vaccines to the injured individual. The cost of providing service to the injured individual (e.g., service cost) may include the expense of providing in-house care, the cost of an assisted living facility, or the cost of putting the injured individual in a nursing home.

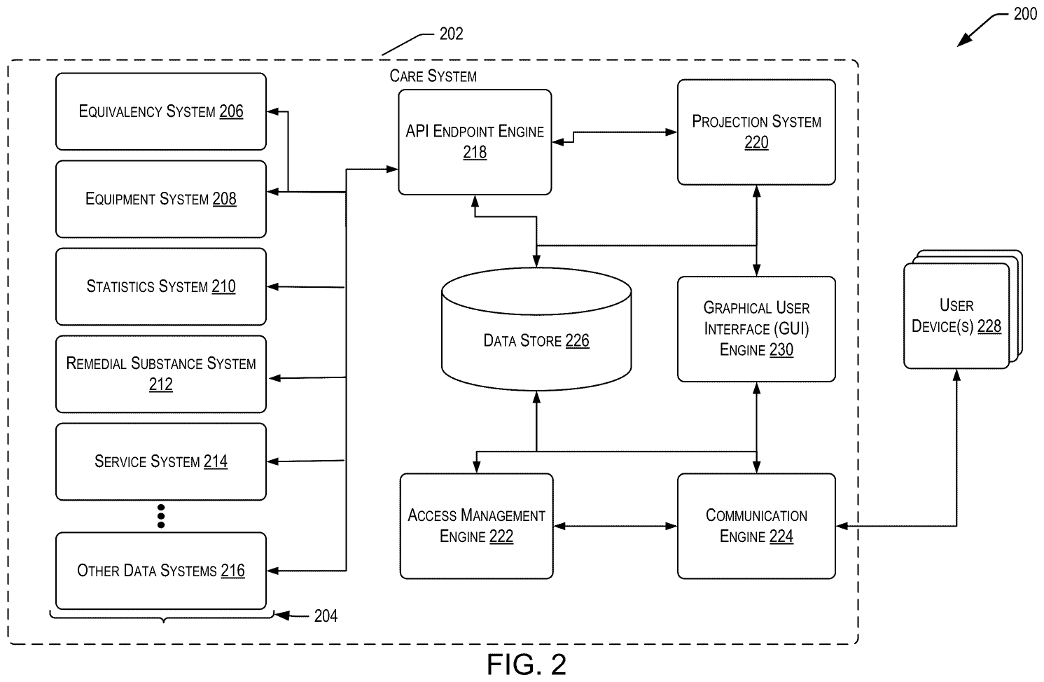

FIG. 2 shows a simplified diagram 200 of a care system according to various embodiments. The diagram 200 includes a plurality of elements connected with directional arrows. The directional arrows not only indicate that the elements are connected, but also indicate the direction that data may flow with respect to the various elements. For example, data may flow between the following elements shown in diagram 200: a user device 228 and a communication engine 224.

Diagram 200 includes a care system 202. The care system 202 is an example of the care system 102 discussed with reference to FIG. 1. The diagram 200 also includes one or more data systems 204. In particular, the one or more data systems 204 include an equivalency system 206, equipment system 208, statistics (e.g., Bureau of Labor Statistics (BLS)) system 210, a remedial substance (a drug cost) system 212, a service system 214, and other data systems 216. The one or more data systems 204 are examples of the one or more data systems 110 discussed with reference to FIG. 1.

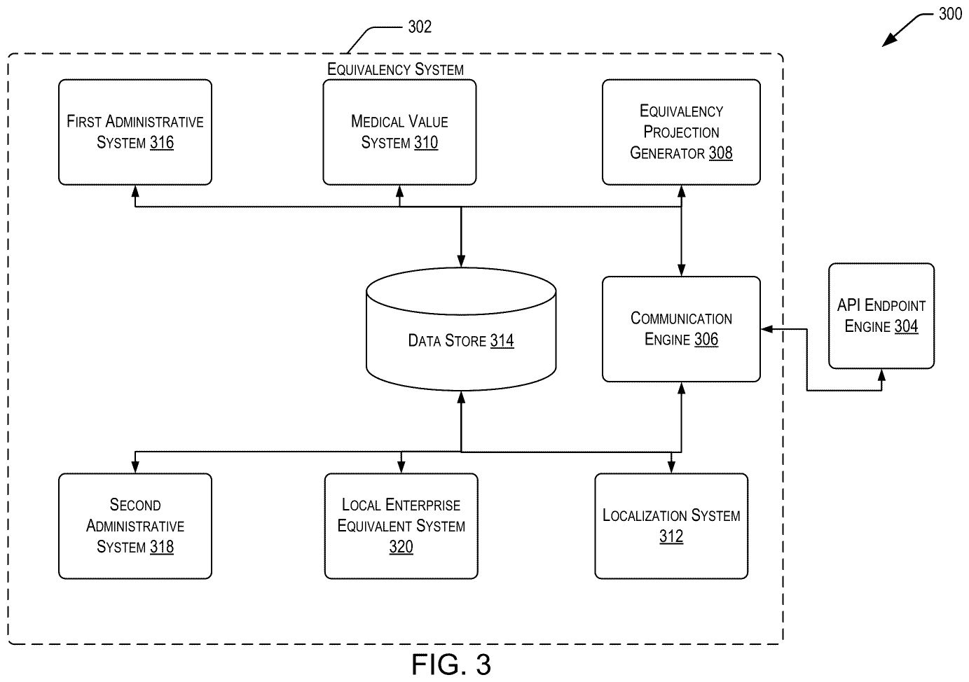

Turning now to the one or more data systems 204, the equivalency system 206 can include any suitable system for calculating an equivalency for a service provided by a medical professional. A equivalency system, according to one embodiment, is described below with reference to FIG. 3. The equipment system 208 can calculate a cost for equipment. The equipment system 208 can receive information identifying a piece of equipment (e.g., a Healthcare Common Procedure Coding System (HCPCS) code) as part of a remediation profile that is input to the system. The remediation profile can be received from a user device 228. The durable medical expense can be calculated by summing a Medicare cost, a enterprise equivalent, a Medicaid cost, a veteran's administration cost, and insurance population data for the equipment. This cost data can be retrieved from a data store in the equipment system 208 using the information identifying the piece of equipment.

Statistics system 210 can maintain a database for each state in the United States. A database can document hourly wages for different occupations in the administrative region corresponding to the database. The database can be searched using information identifying an occupation such as an occupation code or an occupation title. The information can be received from the user device 228 as part of a remediation profile. The user can provide additional information such as an occupation start date, an occupation end date, an annual number of worked hours during a selected duration (e.g., the time between the start data and end date), and an average hourly rate during the selected duration. This information is used to determine a preliminary hourly cost which is multiplied by a markup to obtain the total hourly cost. The markup includes a percentage markup for Social Security's Old-Age, Survivors, and Disability Insurance (OASDI) in addition to a percentage markup for Medicare's Hospital Insurance. The total hourly cost can be multiplied by the hours worked per annum to determine a reasonable value per hour per annum per occupation which can be used to project a reasonable value for lost labor.

Turning now to the remedial substance system 212, the system can locate a drug or vaccine in a drug database using information identifying a particular remedial substance (e.g., drug). This information can be received from a user device 228 as part of a remediation profile and the information can include a market name, a national drug code, a HCPCS code, or a standard nomenclature (e.g., RxNorm concept identifier (CUI), vaccine CVX code). The user can provide a start date for the remedial substance, an end date for a remedial substance, a daily frequency for the remedial substance, and a cost for the remedial substance as part of the remediation profile. The daily frequency and the cost for the remedial substance can be multiplied to determine a daily cost for the remedial substance. This daily price can be multiplied by the length of the service duration (e.g., the time period between the start date and the end date) or for the injured individual's life expectancy (e.g., by multiplying the daily rate, or an annualized rate, by the injured party's life expectancy).

Turning now to the service system 214, the service system can calculate a cost of providing care to an injured individual. Information identifying services related to care can be received from a user device 228 as part of a remediation profile. The information can identify one or more home care services, assisted living services, or nursing services for the injured party. The user can provide a start date for the service, an end date for a service, a daily frequency for a service (e.g., a minimum and maximum number of service units used during the selected duration), and a cost for the service. The daily frequency and the cost for the service can be multiplied to determine a daily cost for the service. This daily price can be multiplied by the length of the service duration (e.g., the time period between the start date and the end date) or for the injured individual's life expectancy (e.g., by multiplying the daily rate, or an annualized rate, by the injured party's life expectancy).

Each of the one or more data systems 204 and the user device(s) 228 may include individual and/or shared storage systems, one or more processors, a user interface, a network connectivity device, and one or more ports. The storage system includes memory that may be implemented, e.g., using magnetic storage media, flash memory, other semiconductor memory (e.g., DRAM, SRAM), or any other non-transitory storage medium, or a combination of media, and can include volatile and/or non-volatile media. The storage systems may also be configured to store computer-executable code or instructions for interacting with the user interface and/or for one or more applications programs, such as an application program for collecting medical-related data generated by the particular generation component.

The one or more processors may be configured to access the operating system and application programs stored within the storage systems and may also be configured to execute such program code. The one or more processors can be implemented as one or more integrated circuits, e.g., one or more single-core or multi-core microprocessors or microcontrollers, examples of which are known in the art. In operation, the one or more processors can control the operation of the particular component. The one or more processors may access and execute the program code and at any given time.

The user interface can include any combination of input and output devices. In some instances, a user can operate input devices of the user interface to invoke the functionality of the particular component or user device. For example, the user interface may enable the user to view, hear, and/or otherwise experience output from a component or a user device via the output devices of the user interface. Examples of output devices include a display, speakers, and the like.

The network connectivity device may enable the component or user device to communicate with the care system 202 and other components or other user devices via one or more networks. The one or more networks may include any suitable combination of cable, cellular, radio, digital subscriber line, or any other suitable network, which may be wired and/or wireless. In some examples, the network connectivity device may enable the component or the user device to communicate wirelessly with various other components and/or the care system 202. For example, the components may include circuitry to enable data communication over a wireless medium, e.g., using near-field communication (NFC), Bluetooth Low Energy, BLUETOOTH® (a family of standards promulgated by Bluetooth SIG, Inc.), Zigbee, Wi-Fi (IEEE 802.11 family standards), or other protocols for wireless data communication.

The care system 202 includes an application programming interface (API) endpoint engine 218, a projection system 220, an access management engine 222, a communication engine 224, and a data store 226. The projection system 220 can use information from the data systems 204 to project a projection for one or more remediation profiles. For example, the projection system 220 can use the information received from the data systems as input to a machine learning model in the projection system 220, and a projection can be provided as output by the model. Generally, the application programming interface (API) endpoint engine 218 is configured to expose endpoints that allow the collection of projection data of different formats generated by software on the one or more data systems 204. The exposed endpoints can also be used to communicate with, or control, software on the one or more data systems 204. The application programming interface (API) endpoint engine 218 may also be configured to perform one or more operations on the collected data. For example, the application programming interface (API) endpoint engine 218 may tag data, log data, perform protocol conversion, and support one-to-many communications. The collection may be asynchronous. In some examples, the projection data has been saved locally in connection with the one or more data systems 204 in many different formats having many different data structures.

From the application programming interface (API) endpoint engine 218, scheduling data flows to the data store 226. The data store 226 (and any other data store discussed herein) may include one or more data stores, which may be distributed throughout two or more different locations (e.g., present on different devices, which can include devices of different entities and/or a cloud server). Depending on the structure of the particular data store, certain data stores may include rules for reading and writing. The data store 226 may include records, tables, arrays, and the like, which may be relational or non-relational. Depending on the data store, scheduling data, records for individual patients, results of clinical studies, business and analytics information, output data from the one or more data systems 204, and the like may be retained. The data within the data store 226 include elements or tags such that a particular data (e.g., individual projection data or a previously generated projection) can be retrieved.

The access management engine 222 is configured to manage access to features of the care system 202, including access to the projection data retained in the data store 226. For example, the access management engine 222 may verify that a user device such as user device(s) 228 is authorized to access the data store 226. To verify the user device(s) 228, the access management engine 222 may require that a user of the user device(s) 228 input a username and password, have a profile associated with the care system, have paid a subscription fee associated with access to the data store 226, and the like. The access management engine 222 may also verify that the user device(s) 228 has an IP address or geographical location that corresponds to an authorized list, that the user device(s) 228 includes a plug-in for properly accessing the data store 226, that the user device(s) 228 is running certain applications required to access the data store 226, and the like.

The communication engine 224 is configured to retrieve the data from the data store 226 and provide one or more interfaces for interacting with elements of the care system 202. For example, the communication engine 224 includes an interface by which an application running on user device(s) 228 can access portions of data within the data store 226 communication engine 224 can be a version of communication engine 106 described above.

The graphical user interface (GUI) engine 230 can generate a graphical user interface that is provided to the user device(s) 228. The graphical user interface can include text fields where a user can input the information that is provided to the data systems 204. The graphical user interface can be used to present a map that the user can interact with to select a administrative location. This map can be used to dynamically calculate remediation profiles or life expectancy projections according to embodiments of the present disclosure. The graphical user interface engine 230 can be used to generate the graphical user interfaces 500-800 described below.

FIG. 3 shows a simplified diagram 300 of an equivalency system according to at least one embodiment. The diagram 300 includes a plurality of elements connected with directional arrows. The directional arrows not only indicate that the elements are connected, but also indicate the direction that data may flow with respect to the various elements. For example, data may flow between the following elements shown in diagram 300: a communication engine 306 and an API endpoint engine 218.

The equivalency system 302 can communicate with an API endpoint engine 218 via a communication engine 306. The API endpoint can correspond to the API endpoint engine 218 described above with reference to FIG. 2. For instance, the equivalency system 302 can receive a request for a reasonable value for a service via the communication engine 306. The request can include a personal profile and a remediation profile. After calculation, a response with information identifying a reasonable value for the service can be provided to the API endpoint engine 218 via the communication engine 306. The request can include information identifying a service as part of the remediation profile, and, for instance, the information can be a healthcare common procedure coding system (HCPCS) code for the service and a personal profile for an injured party associated with the service (e.g., the recipient of the service).

The request can be provided to a equivalency projection generator 308 which can use the information in the request to retrieve relevant information from within the equivalency system. For instance, a reasonable value for a service can be calculated by summing a service's first equivalency (e.g., Medicare cost), enterprise equivalent, a first administrative remediation unit (e.g., a first jurisdictional medical cost) (e.g., Medicaid cost), a second administrative remediation unit (e.g., a second jurisdictional medical cost) (e.g., Veteran's Administration cost), and uncovered equivalent (e.g., the uninsured cost). These values may be multiplied by weights before they are summed to calculate the reasonable value for the service.

The first equivalency can be retrieved from a equivalency system 310. This first equivalency is a Medicare value that is set at a national level, but the equivalency is projected for a specific location that is identified in the request. Accordingly, the request can identify a location where the service will be provided. The first equivalency can be localized by multiplying the first equivalency by a local weight to produce a weighted first equivalency. The local weight can be obtained from a localization system 312. The first equivalency, and the weight, can be retrieved from databases in the equivalency system 310 and the localization system 312 respectively, or the value and weight can be retrieved from data store 314. The equivalency system 310 can provide the first value to the equivalency projection generator 308, and the local weight can be provided to the equivalency projection generator 308 by the localization system 312. The equivalency projection generator 308 can multiply the first equivalency by a local weight to generate a weighted first equivalency.

The first administrative equivalency and the second administrative equivalency can be retrieved by the first administrative system (e.g., the first jurisdictional medical system) 316 and the second administrative system (e.g., the second jurisdictional medical system) 318 respectively. The first administrative system 316 can maintain a Medicaid database for each state in the United States, and the second administrative system can maintain a Veteran's Administration database for each state. The databases may be part of each system, or the databases can be located in data store 314. In response to the request, the first administrative equivalency can be retrieved (e.g., from a database in the system or from the data store 314) by the first administrative system 316 and provided to the equivalency projection generator 308. The second administrative equivalency can be retrieved by the second administrative system 318 and provided to the equivalency projection generator 308.

The enterprise equivalent for the service can be retrieved by a local enterprise equivalent system 320 and provided to the equivalency projection generator 308. The local enterprise equivalents can be a markup that is added to the weighted first equivalency (e.g., the localized Medicare cost). The enterprise equivalent varies by state and a commercial database, either in the local enterprise equivalent system 320 or the data store 314, stores local values for the cost markup. The local enterprise equivalent system 320 can use the location in the request to retrieve the local enterprise equivalent and provide the cost to the equivalency projection generator. In addition, the uncovered equivalent can be received from a user device (e.g., user device 228) via the API endpoint engine 218. The request can also include a start date, an end date, a frequency, and a service cost (e.g., a minimum cost, a maximum cost, an average cost).

The equivalency projection generator 308 can use the first equivalency (e.g., Medicare cost), enterprise equivalent, first administrative remediation unit (e.g., Medicaid cost), second administrative remediation unit (e.g., Veteran's Administration cost), and uncovered equivilant to calculate an equivalency for a service. These values may be summed by the equivalency projection generator 308 to produce an equivalency for the service identified in the request. In some embodiments, the values may be multiplied by weights before summing. The equivalency projection generator 308 can determine a weight for each value, or the same weight can be assigned to each value.

Information from the request for an equivalency for a service can be used to calculate the equivalency value. For example, the frequency can be used to determine a number of equivalencies for a period of time (e.g., a day, a week, a month, a year). For example, the equivalency can be multiplied by the frequency to produce a equivalency rate. The request may include a life expectancy for the injured individual, and the equivalency rate can be multiplied by the remaining life expectancy for the injured individual to determine a number of equivalencies over the life of the individual. For example, if the individual is 60 with a 260-year life expectancy and the reasonable value rate is 20 equivalencies/year, the equivalencies over the injured individual's life expectancy are 500 equivalencies (e.g., 20 years×20 equivalencies/year).

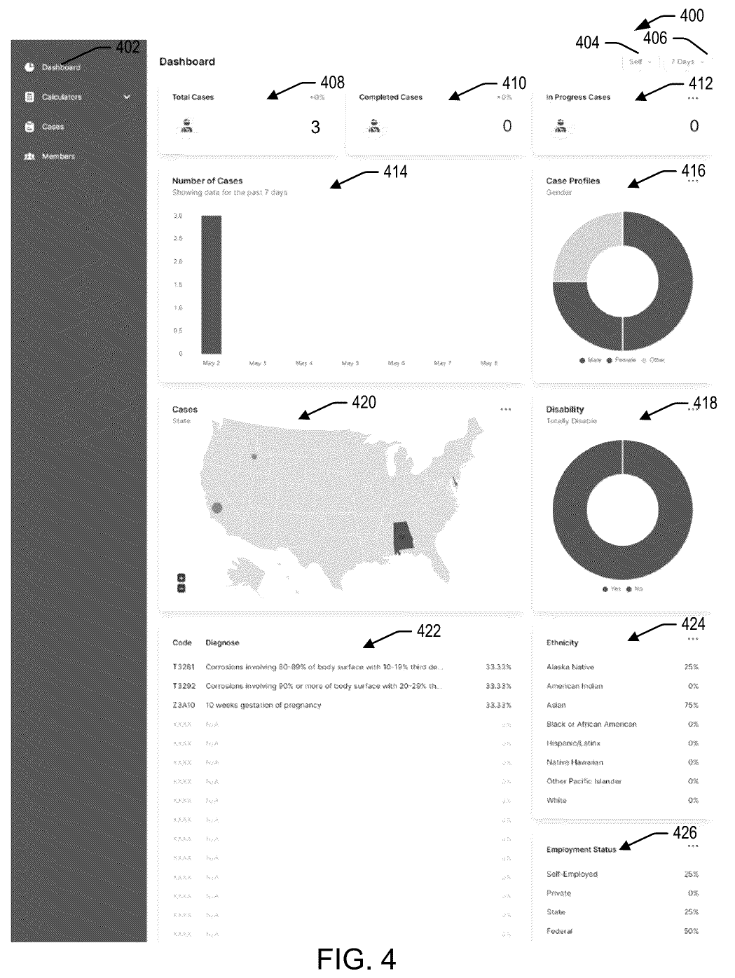

FIG. 4 shows a graphical user interface with a dashboard 400 according to at least one embodiment. Any of the graphical user interfaces in this disclosure can be executed by a user device. The graphical user interfaces can be implemented as a web browser application or an independent software application. The dashboard 400 can display a menu for navigating system(s) associated with the interface, and summary statistics about the projections generated by the system. The dashboard 400 shown in the graphical user interface can be presented in response to a user selecting graphic 402. The dashboard 400 includes a configurable set of windows that each present statistics about projections for particular cases.

The specific cases that are used to generate the statistics shown in dashboard 400 can be configured with field 404 and field 406. The specific cases can be associated with an account and field 404 can be used to select one or more accounts. The system associated with dashboard 400 can use the cases associated with the selected accounts as sources for the statistics shown in the dashboard. In addition, field 406 can be used to specify a time period for the statistics. As an example, field 406 shows “7 days” which means that the statistics in the dashboard were generated from cases that were generated in the last week. Other time periods are contemplated, and any conceivable time periods can be selected.

The statistics about cases are displayed in windows within dashboard 400. These windows can display information about cases in any format such as charts, graphics, maps, or tables. For instance, window 408 shows a count of the total number of cases generated by the account specified in 404 during the time period specified in 406. Similarly, window 410 shows the number of cases shown in window 408 that have been completed, and window 412 shows the number of cases shown in window 408 that are in-progress.

The windows can display charts or other graphics that are created using the cases. For instance, window 414 shows a bar graphic with the daily count of cases created over the last week. Window 416 shows a pie chart of the breakdown of cases by gender, while window 418 displays a pie chart with the number of cases that resulted in total disability for the subject. In addition, window 420 shows a map that indicates the locations for each case that conforms to the parameters entered using field 404 and field 406.

The windows can also display tables for the cases selected using field 404 and field 406. For example, window 422 shows a table with the diagnosis for the cases, window 424 shows a table with the ethnicity of the subjects of the cases, and window 426 shows a table with the employment status for the subjects. These windows, and the windows mentioned above, can be configured to display any information requested by a user. The settings for the graphical user interface can be used to configure the order and orientation for the windows.

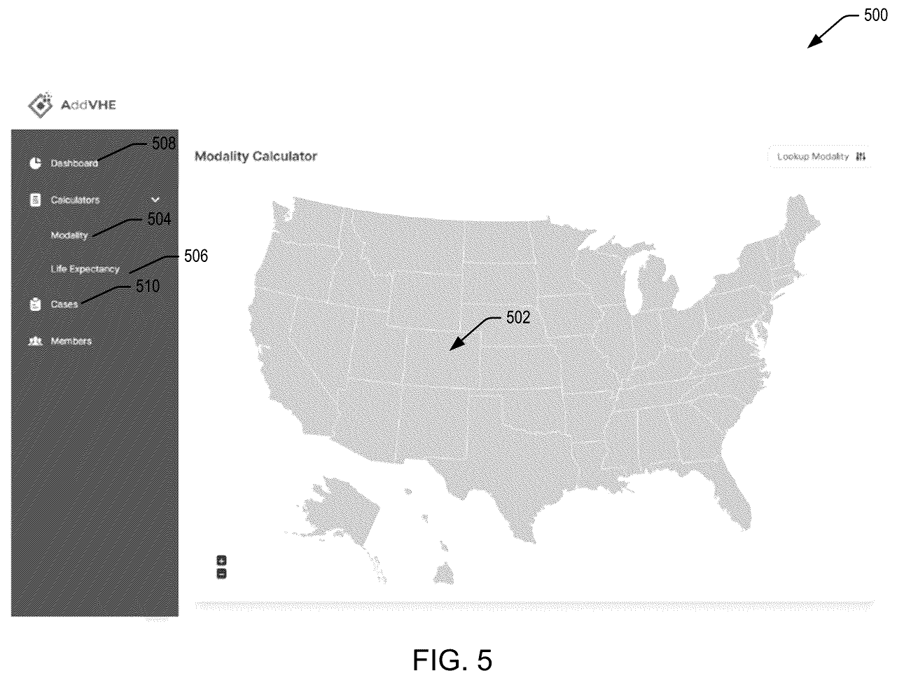

FIG. 5 shows a graphical user interface 500 according to at least one embodiment. Graphical user interface 500 includes an interactive map 502 that a user can interact with to administrative regions. The interactive map 502 can be used in conjunction with a calculator such as a remediation preference calculator or a life expectancy calculator. Selecting graphic 504 can initiate a remediation preference calculator workflow that can allow the interactive map 502 to be used to display different projections. Similarly, graphic 506 can begin a life expectancy workflow that can allow the interactive map to be used to display different life expectancy projections. In addition, graphic 508 can display a dashboard with summary statistics for any cases associated with the user profile that are associated with the user device that is displaying graphical user interface 500. Graphic 510 can display a case summary page that shows a detailed depiction of the cases associated with the profile. Selecting graphic 504 can cause the user device to display graphical user interface 600 and selecting graphic 506 can cause the user device to display graphical user interface 800.

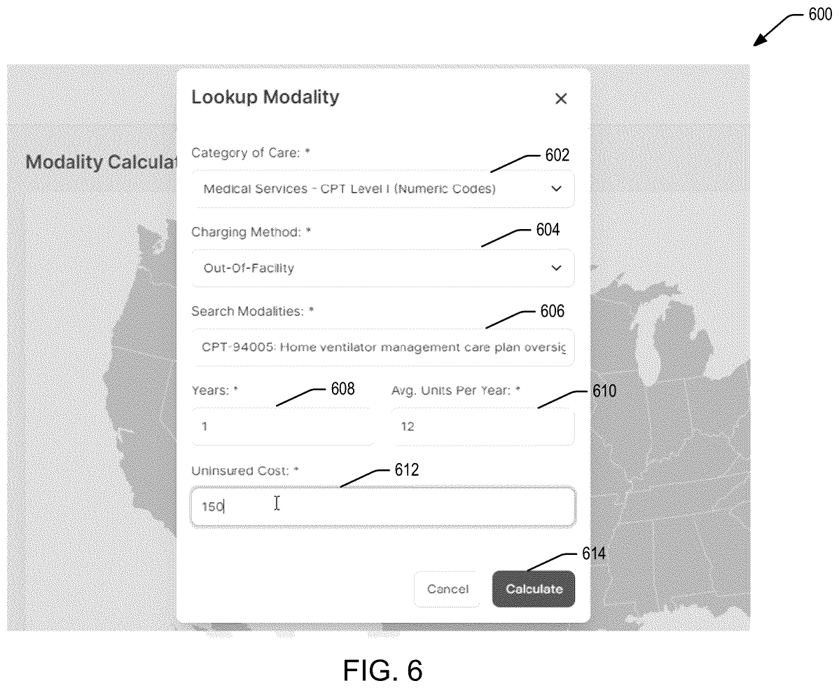

FIG. 6 shows a graphical user interface 600 with a remediation preference calculator according to at least one embodiment. Graphical user interface 600 can be used to provide information that forms part of a remediation profile. For instance, field 602 can be used to collect a category of remediation (e.g., a category of care), field 604 can be used to obtain a remedial location designation (e.g., a charging method), field 606 can be used to collect a search remediation preference, field 608 can collect a number of years for the remediation preference, field 610 can be used to provide an annual frequency of units for the remediation preference, and field 612 can allow a user to provide an uncovered equivalent for the remediation preference. After the remediation profile has been entered, selecting graphic 614 can be used to calculate the remediation profile. Additional information can be used to calculate the remediation profile such as a personal profile associated with a current user or case. Selecting graphic 614 can cause graphical user interface 700 to be displayed on the user device.

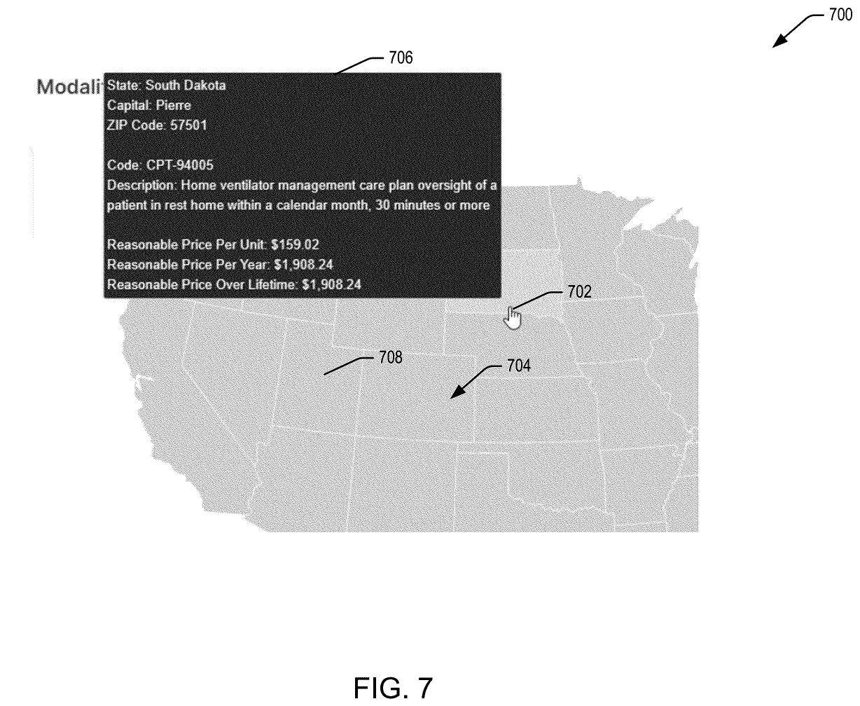

FIG. 7 shows a graphical user interface 700 with an interactive map and remediation preference calculator according to at least one embodiment. Cursor 702 can be used to dynamically select a region for a projection. By changing the position of cursor 702 on interactive map 704, the projection, shown in windows 706, can be updated. As shown in graphical user interface 700, cursor 702 is on South Dakota and the projection shown in window 706 reflects data from that location. If the cursor was to move to position 708 on Utah, the projection in window 706 would be updated for that location. While interactive map 704 allows a user to select states, other administrative regions are contemplated. For example, interactive map 704 could allow a user to select cities, states, regions of states, zip codes, or any other geographical unit. While graphical user interface 700 shows a remediation profile in window 706, the graphical user interface could be used to dynamically update a life expectancy projection (e.g., using the information collected by graphical user interface 800).

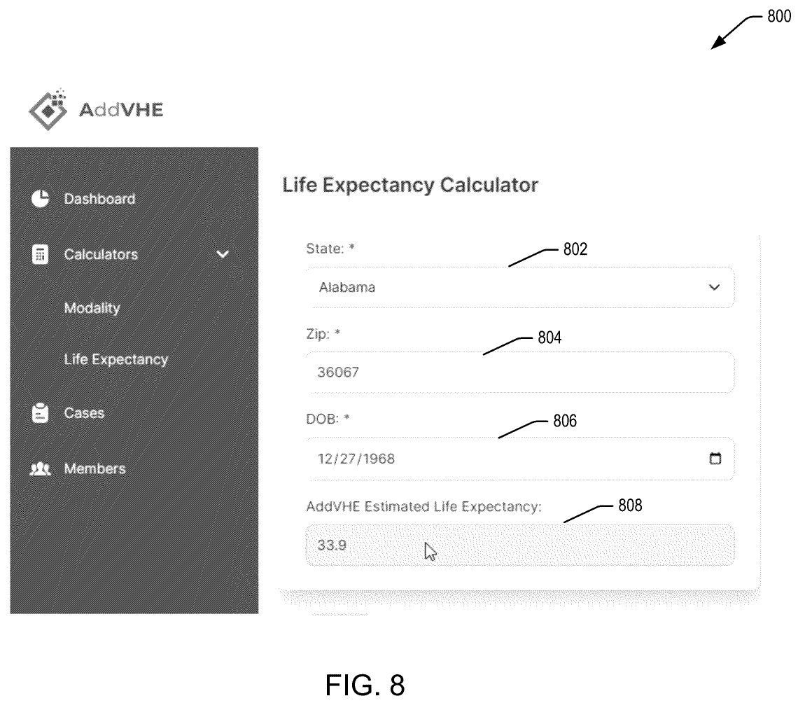

FIG. 8 shows a graphical user interface 800 with a life expectancy calculator according to at least one embodiment. Graphical user interface 800 can be used to collect information that can be used to calculate a life expectancy for an individual. The information can be part of a remediation profile. Graphical user interface 800 can be displayed in response to a user selecting graphic 506 in graphical user interface 500.

Field 802 can be used to input a state where the individual who is having their life expectancy calculated currently resides. Field 804 can be used to input a current zip code where the individual resides. If the life expectancy projection is used in conjunction with an interactive map, as shown in graphical user interface 700, the information from field 802 and 804 may be updated based on inputs to graphical user interface 700. A birth date for the individual can be input to field 806 as part of the remediation profile. The remediation profile input to graphical user interface 800, and a personal profile for the individual, can be used to calculate the estimated life expectancy shown on field 808. This life expectancy can be calculated from the current date and show the number of additional years that the subject of the profile is expected to live. This life expectancy can depend on the individual's diagnosis and the individual's current treatment.

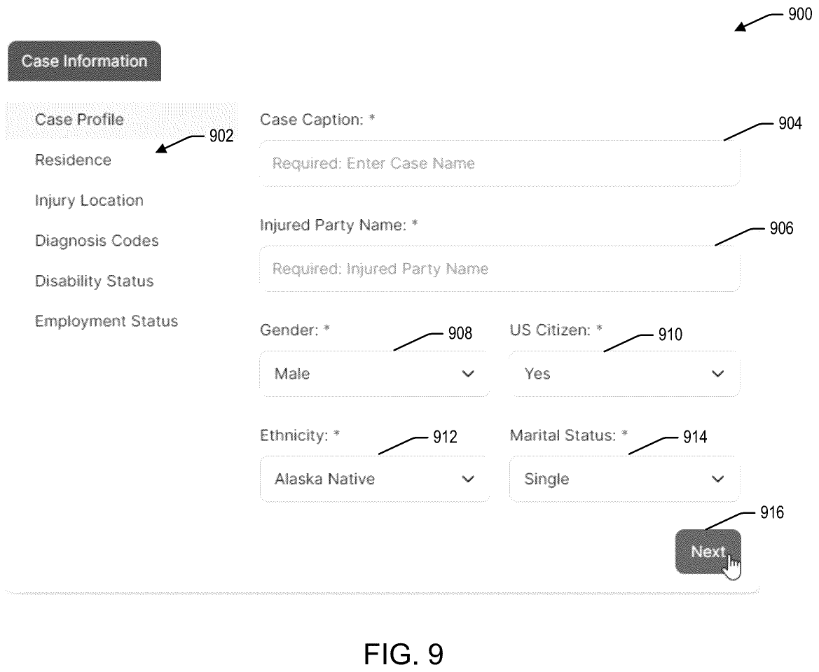

FIGS. 9-14 show various interfaces for building a case profile, according to various embodiments. The case profile can be used to project the cost of caring for an individual who is the subject of the profile. This cost can be contextual and the cost can vary by location because of differences in the annual cost of treatment as well as differences in life expectancy. The case profile can highlight these differences so that an informed decision about the individual's care can be made. FIG. 9 shows a graphical user interface 900 for entering personal (e.g., demographic) information for a case profile according to at least one embodiment. Graphical user interface 900 can be used to enter a case profile as part of providing the case information. The case information can be entered using graphical user interfaces 900-1400 and these user interfaces can be navigated by selecting graphics in menu 902. In addition, graphic 916 can be selected to navigate to graphical user interface 1000.

Information for the case can be entered into the fields within graphical user interface 900. This information, and the information entered in graphical user interfaces 1000-1400, can be used to prepare a projection or for use in any of the techniques disclosed herein. For instance, a name for the case profile can be entered using field 904. The name can be an informal name for the case so that the case can be identified by the user creating the profile. Field 906 can be used to enter a name for the injured party (e.g., the subject). Additional fields can be used to enter personal information about the subject. For instance, the subject's gender can be entered in field 908, the subject's citizenship status can be added to field 910, the subject's ethnicity can be entered in field 912, and the subject's marital status can be entered at 914.

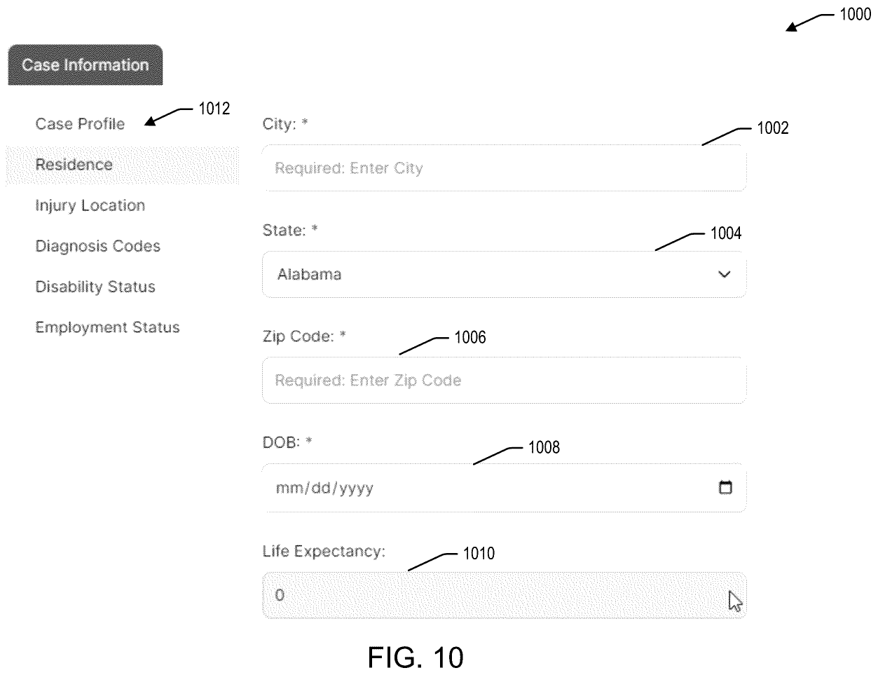

FIG. 10 shows a graphical user interface 1000 for entering residence information for a case profile according to at least one embodiment. The residence information can be for the subject, and the city where the subject currently resides can be entered at 1002. The state where the subject resides can be entered in 1004 and the subject's zip code can be entered at 1006. In some embodiments, one of fields 1002-1106 can be entered and the system can auto-populate the remaining fields. In addition, a date of birth for the subject can be entered using field 1008.

The information entered in fields 1002-1008 can be used to calculate a life expectancy that is displayed in field 1010. In addition, information entered in graphical user interfaces 900 and 1100-1400 can be used to calculate the life expectancy. For instance, the gender, ethnicity, and marital status entered via graphical user interface 900 and the information from graphical user interface 1000 can be used to look up a life expectancy projection from a database. The information may be provided as input to a machine learning model that can output a life expectancy projection for the subject. Graphical user interface 1000 can also include a menu 1012 that can be used to navigate between graphical user interfaces 900-1400.

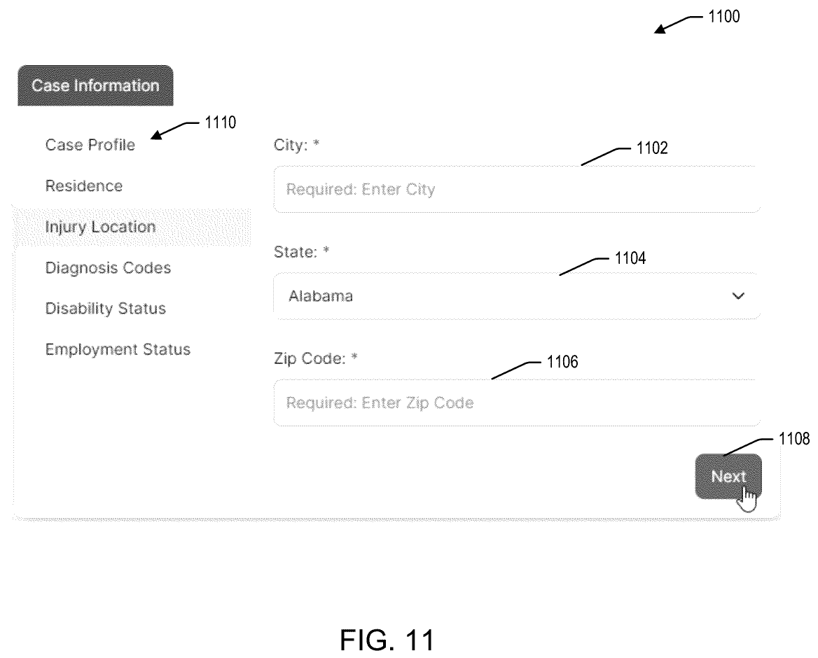

FIG. 11 shows a graphical user interface 1100 for entering information about an injury location for a case profile according to at least one embodiment. A city where the subject was injured can be entered at 1102, a state where the subject was injured can be entered at 1104, and a zip code where the subject was injured can be entered at 1106. The graphical user interface 1200 can be selected by interacting with graphic 1108. In addition, graphical user interfaces 900-1400 can be navigated using the menu 1110.

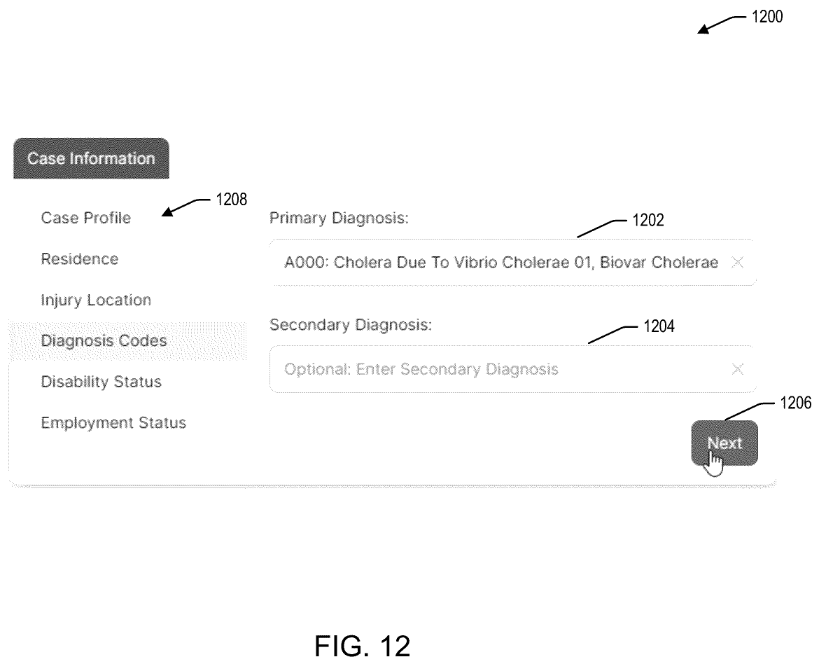

FIG. 12 shows a graphical user interface 1200 for entering information about an injury diagnosis for a case profile according to at least one embodiment. A primary diagnosis can be entered at field 1202 and a secondary diagnosis can be entered at field 1204. The diagnosis can be entered using keyword search or a diagnostic code, and/or by providing any other information identifying a diagnosis. In addition, graphical user interfaces 900-1400 can be navigated using the menu 1208

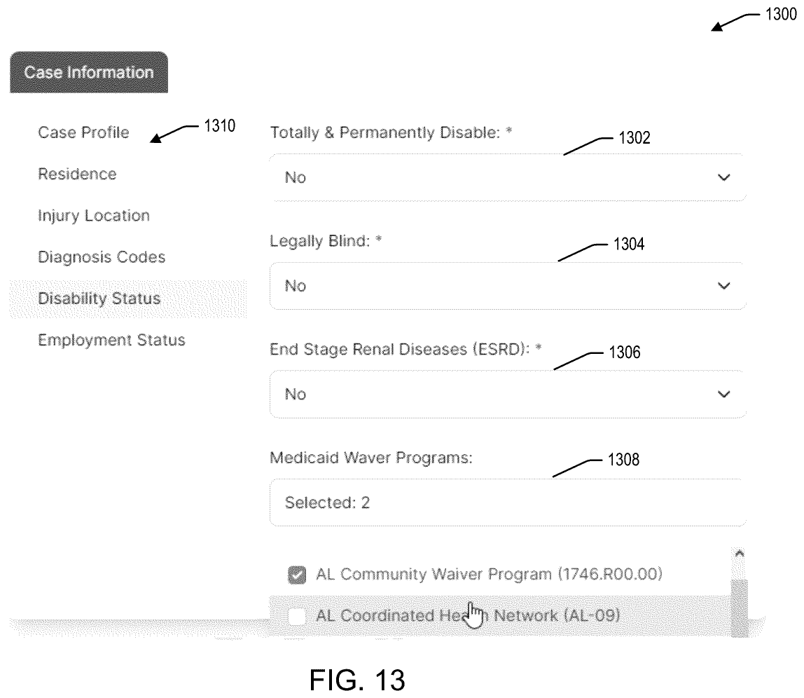

FIG. 13 shows a graphical user interface 1300 for entering information about a subject's disability status for a case profile according to at least one embodiment. Whether the subject is totally and permanently disabled can be entered at field 1302, whether the subject is legally blind can be entered at field 1304, and whether the subject is experiencing end stage renal disease (ESRD) can be provided at field 1306. Any Medicare waver programs for the subject can be entered at field 1308. In addition, graphical user interfaces 900-1400 can be navigated using the menu 1310.

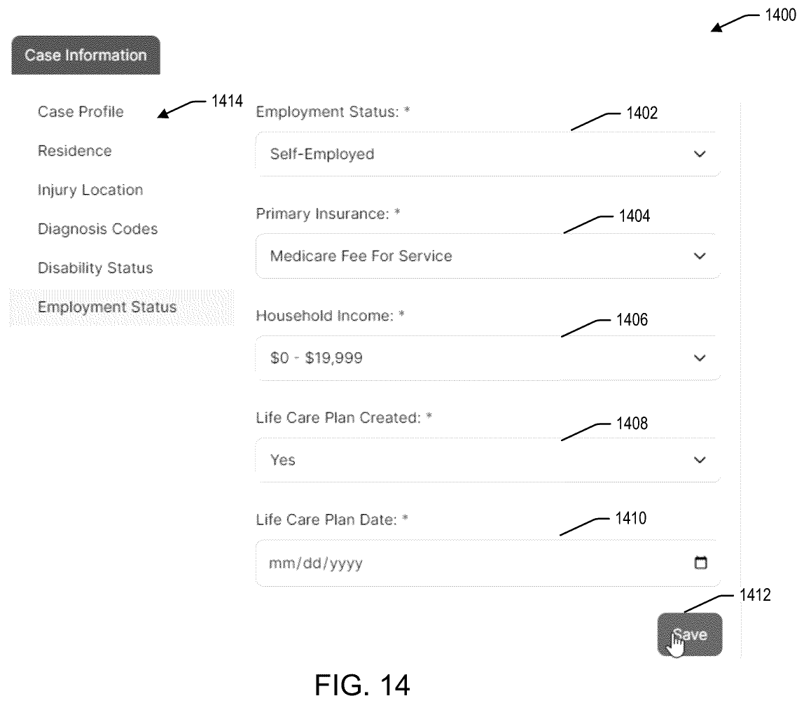

FIG. 14 shows a graphical user interface 1400 for entering information about a subject's employment status for a case profile according to at least one embodiment. The employment information can include financial information related to the subject. Field 1402 can be used to provide the subject's employment status (e.g., employed, unemployed, self-employed, retired, etc.). Field 1404 can be used to provide the subject's primary insurance, and field 1406 can be used to provide the subject's household income.

Field 1408 can be used to indicate whether the subject has a life remediation profile and field 1410 can be used to indicate a start date for the life remediation profile. A life remediation profile can be a report summarizing the subject's current care needs as well as any anticipated future care needs. The remediation profile can be used as evidence in personal injury litigation in some jurisdictions, and the remediation profile can be saved by selecting graphic 1412 In addition, graphical user interfaces 900-1400 can be navigated using the menu 1414.

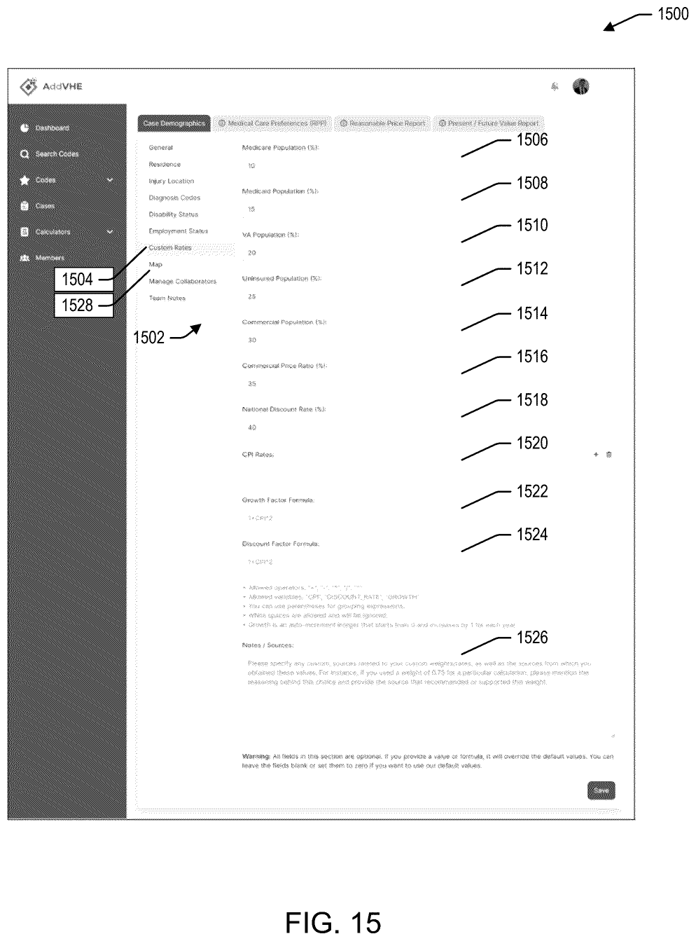

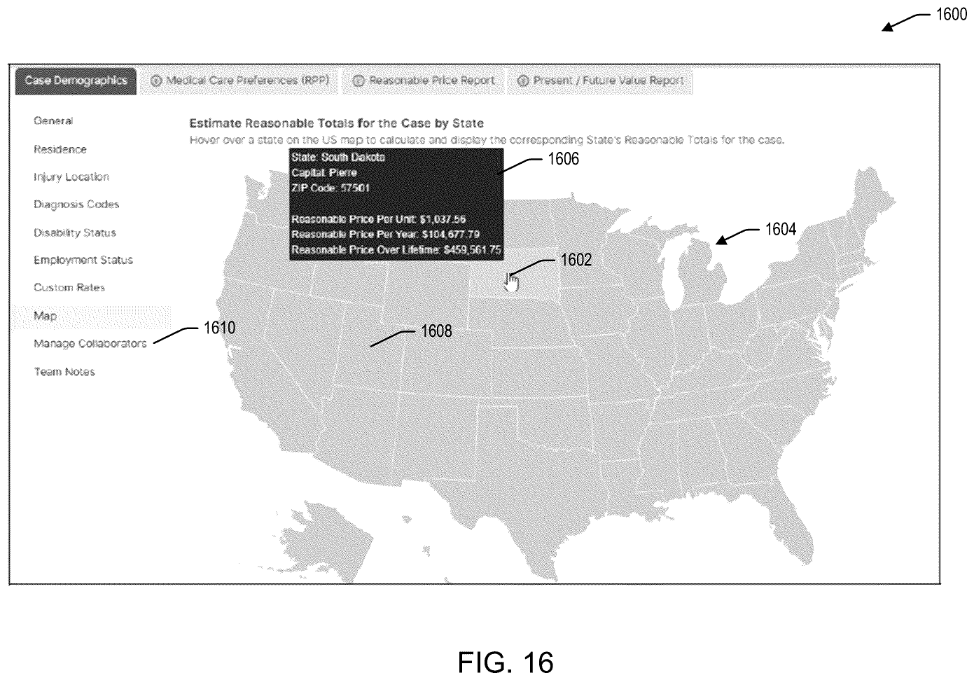

FIG. 15 shows a graphical user interface 1500 for entering custom values for a projection. Once the initial information has been entered for a projection, the system can present additional fields in the menu 1502 that can be used to access graphical user interfaces 15-23. For instance, graphical user interface 1500 can be accessed by selecting graphic 1504. In addition, graphical user interface 1600, described below with reference to FIG. 16, can be accessed using graphic 1528.

Graphical user interface 1500 can be used to enter custom percentages and weights for a projection. For instance, field 1506 can be used to customize the Medicare population percentage (e.g., the percentage of the population receiving Medicare benefits). Field 1508 can be used to specify the percentage of the population receiving Medicaid benefits. Field 1510 can be used to specify the percentage of the population receiving Veterans' Administration population. Field 1512 can be used to specify the percentage of the population that are uninsured. Field 1514 can be used to specify the percentage of the population that are part of the commercial population. Field 1516 can be used to specify the commercial price ratio, field 1518 can be used to specify the national discount rate, and field 1520 can be used to specify the consumer price index (CPI) inflation rate. The formulas used to calculate the projection can be specified using graphical user interface 1500. For instance, field 1522 can be used to specify a growth factor formula and field 1524 can be used to specify a discount factor formula. In addition, field 1526 can be used to provide notes including references or sources for the rates, percentages, weights, and formulas that were entered via graphical user interface 1500.

FIG. 16 is a graphical user interface 1600 for comparing reasonable totals by state according to at least one embodiment. Cursor 1602 can be used to dynamically select a region for a projection. By changing the position of cursor 1602 on interactive map 1604, the reasonable totals shown in windows 1606, can be updated. The reasonable totals can be the fair market value for a product or service in a given administrative region (e.g., state and zip code). As shown in graphical user interface 1600, cursor 1602 is on South Dakota and the projection shown in window 1606 reflects data from that location. If the cursor was to move to position 1608 on Utah, the reasonable total in window 1606 would be updated for that location. While interactive map 1604 allows a user to select states, other administrative regions are contemplated. For example, interactive map 704 could allow a user to select cities, states, regions of states, zip codes, or any other geographical unit.

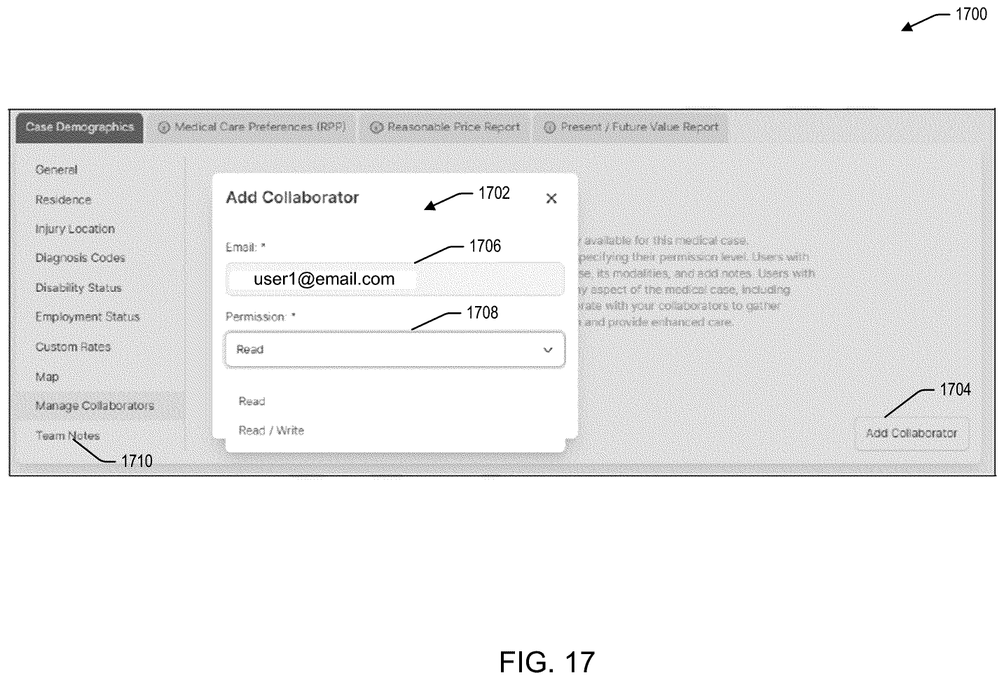

FIG. 17 shows a graphical user interface 1700 for adding collaborators to a remediation profile according to at least one embodiment. A user can open a window 1702 with options for inviting an additional user to join a collaboration team by selecting graphic 1704. Field 1706 can be used to identify an additional user that is invited to join the collaboration team and collaborate on the case. The additional user can be identified with an email address, a phone number, a user identifier, an account name, an account number, or any other identifying information. An invitation with a link and instructions on how to join the collaboration can be sent to the additional user using the information in field 1706. An additional user that is invited to collaborate on a case can be added to the case's collaboration team using the instructions and link. The case's collaboration team can include all users with permission to view or modify the case.

Field 1708 can be used to set the permission for the additional user. The permissions can be set to “read” which allows the additional user identified in field 1706 to read the one or more remediation profiles prepared for the case. The permission in field 1708 can be set to “read/write” and the additional user can be allowed to create, modify, or delete one or more remediation profiles in the case. Users, once added to the team, can share notes about the case using graphical user interfaces 1800-1900. These notes, and information about the care team, can be viewed by selecting graphic 1710 and accessing graphical user interface 1900.

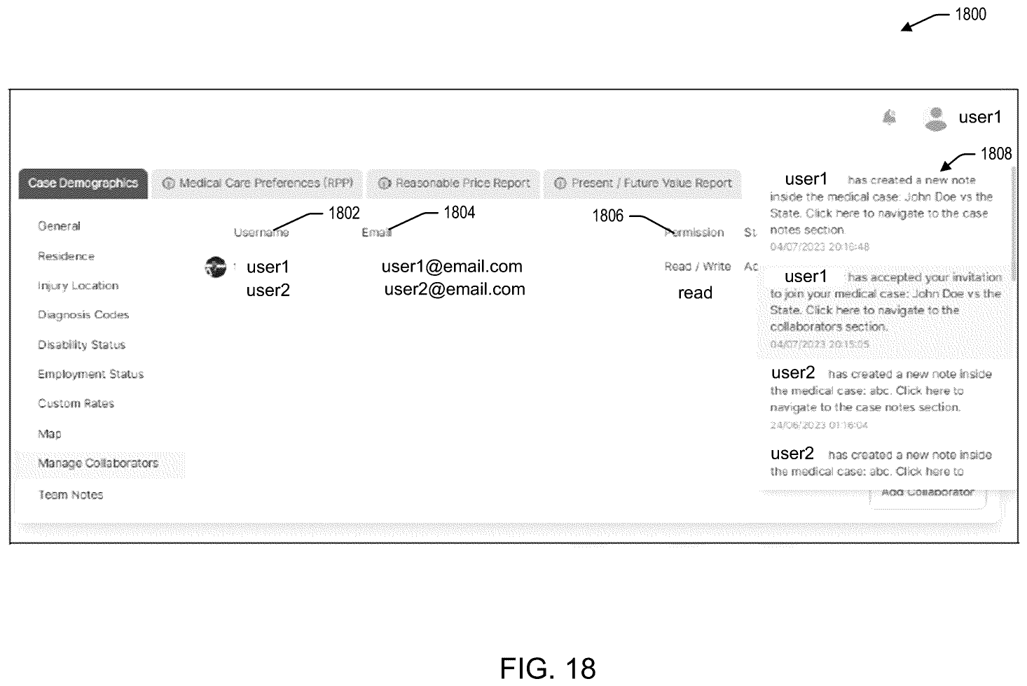

FIG. 18 shows a graphical user interface 1800 that displays information about a collaboration team according to at least one embodiment. Field 1802 shows the usernames for the current members of the collaboration team. Field 1804 shows identifying information for the collaboration team members. The collaboration team members can be identified with an email address, a phone number, a user identifier, an account number, or any other identifying information. Field 1806 shows the assigned permission for each member of the collaboration team. Messages can be exchanged between users and these messages are displayed on window 1808. Messages may be generated in response to events “User 1 has accepted your invitation” or the messages may include written notes. Notes can be created using graphical user interface 1900.

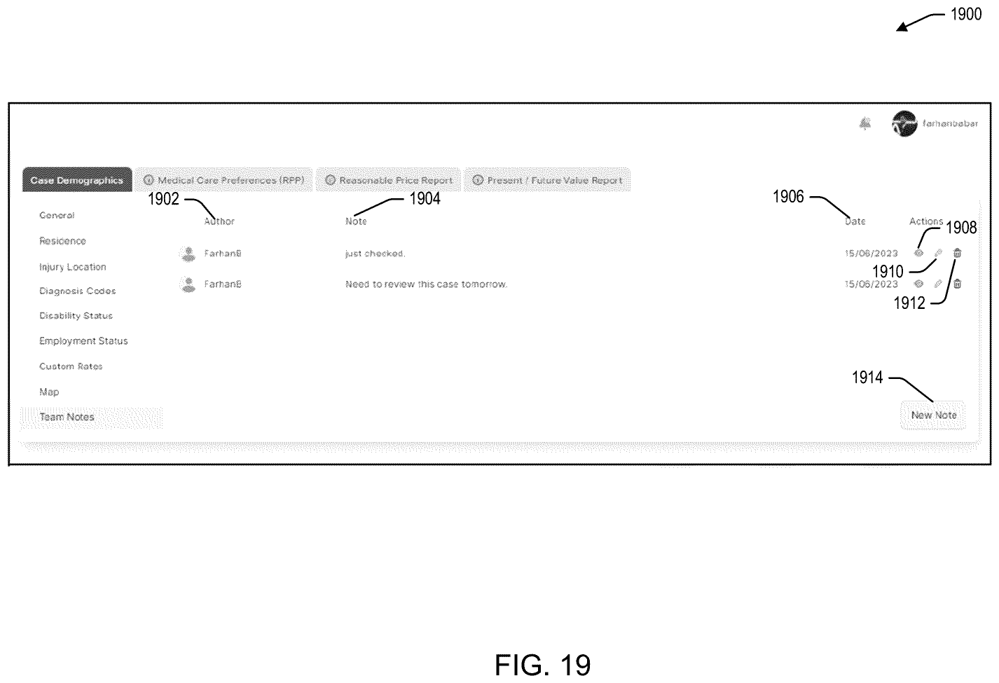

FIG. 19 shows a graphical user interface 1900 that displays notes shared with a collaboration team according to at least one embodiment. Field 1902 shows the author for each note and 1904 shows a title or text from the note. Field 1906 shows the date when the corresponding note was created. The full note text, and any attachments, can be viewed by selecting graphic 1908. The note can be edited by selecting graphic 1910, and the note can be deleted by selecting graphic 1912. A new note can be created by selecting graphic 1914 which can cause graphical user interface 2000 to be displayed.

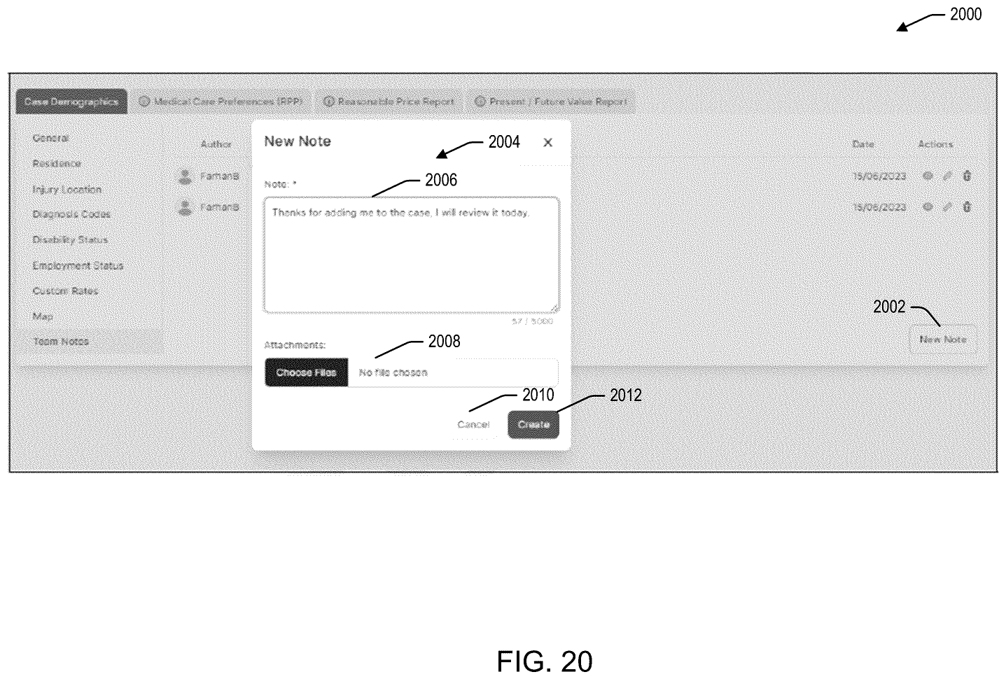

FIG. 20 shows a graphical user interface 2000 for drafting a note according to at least one embodiment. Graphic 2002 can be selected to display window 2004 which can be used to draft notes. Field 2006 can be used to input text-based messages that are added to the note and field 2008 can be used to upload files that are included in the note. Graphic 2010 can be selected to close window 2004 without sharing the message with the team and graphic 2012 can be used to post the message so that the message is shared with the collaboration team.

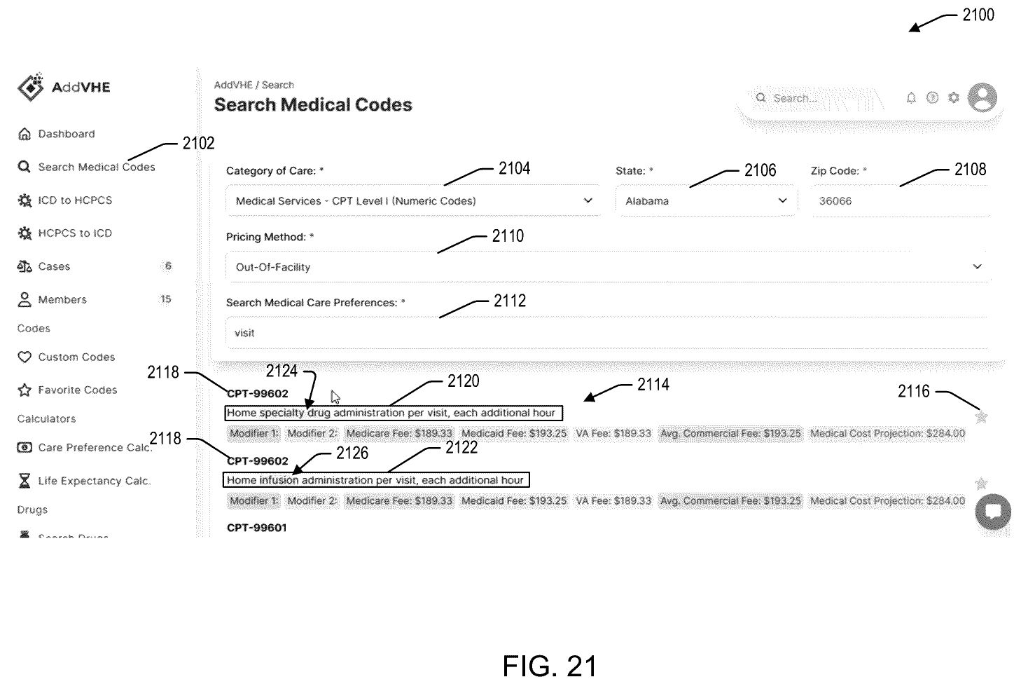

FIG. 21 shows a graphical user interface 2100 for searching for search modalities according to at least one embodiment. The search modalities can include information identifying remedial preferences such as medical codes such as a Healthcare Common Procedure Coding System (HCPCS) code, RxNorm concept identifier (CUI), vaccine CVX code, diagnostic code, or any other numeric or alphanumeric code that can be used to identify medical information. The graphical user interface 2100 can be displayed in response to a user selecting graphic 2102. The category of code that is to be searched for can be specified in field 2104. These categories can include medical services, equipment, transportation, and labor. The state corresponding to the desired code can be specified in field 2106, and the zip code corresponding to the search can be specified in field 2108. A remedial location designation for the search can be specified in field 2110. These remedial location designations can include an out of facility or an in facility remedial location designation.

Search modalities can be provided in field 2112. These search modalities can include information identifying remedial preferences such as a medical code, a keyword search, or a plan text search using natural language. For example, the search modalities can include the product name, the item name, the service name, the Current Procedural Terminology (CPT) code, the Healthcare Common Procedure Coding System (HCPCS) code, the National Drug Code (NDC) code, or any other search modality. The results returned in response to the search modalities (e.g., search results) can be displayed in window 2114. The results can be organized by relevance where results that most closely match the search modality are shown before results that less closely match the search modality.

The relevance of search results can be determined by comparing the information identifying a category of remediation input in field 2112 (e.g., a search modality) and a description for each remediation preference using any appropriate search algorithm. A remediation preference can correspond to multiple different versions with different descriptions, and, for example, medical remediation preference 2118 can include a first description 2120 and a second description 2122. Determining the appropriate version of a remediation preference by visually comparing the description (e.g., first description 2120 and second description 2122) can be tedious and time consuming because the differences between the descriptions may not be easily apparent to a user. Graphical user interface 2100 can visually indicate the differences between the description by comparing the descriptions for the versions of a remediation preference that are returned as search results and the user interface can visually identify differences between the descriptions. This visual highlighting offers a technical improvement by making it easier for visually impaired users to navigate the user interface. For example, the different versions of a remediation preference in the search results can be displayed sequentially and the differences in the descriptions (e.g., first description 2120 and second description 2122) can be indicated by graphic 2124 and graphic 2126. In other embodiments, the text of the descriptions that is the same can be visually indicated in addition or alternatively to the visual indication of the different language in the descriptions. Such visual indications can reduce the storage or communication requirements of a system implementing the user interface, because fewer errors will result in fewer corrections to a projection, and fewer stored versions of the projection.

In addition, the results can be organized by cost from lowest cost to highest cost or highest cost to lowest cost. The cost can be the Medicare fee, the Medicaid fee, the Commercial fee, or the cost projection (e.g., the care projection). The cost projection can be a value calculated from the Medicare fee, the Medicaid fee, and the Commercial fee. Displaying multiple fees can help a user interacting with graphical user interface 2100 to understand how the projection is calculated. Displaying the fees can allow the user to determine which search result to select and whether to create a custom code as described with reference to graphical user interface 2400. One or more of the search results can be added to a starred code list by selecting graphic 2116.

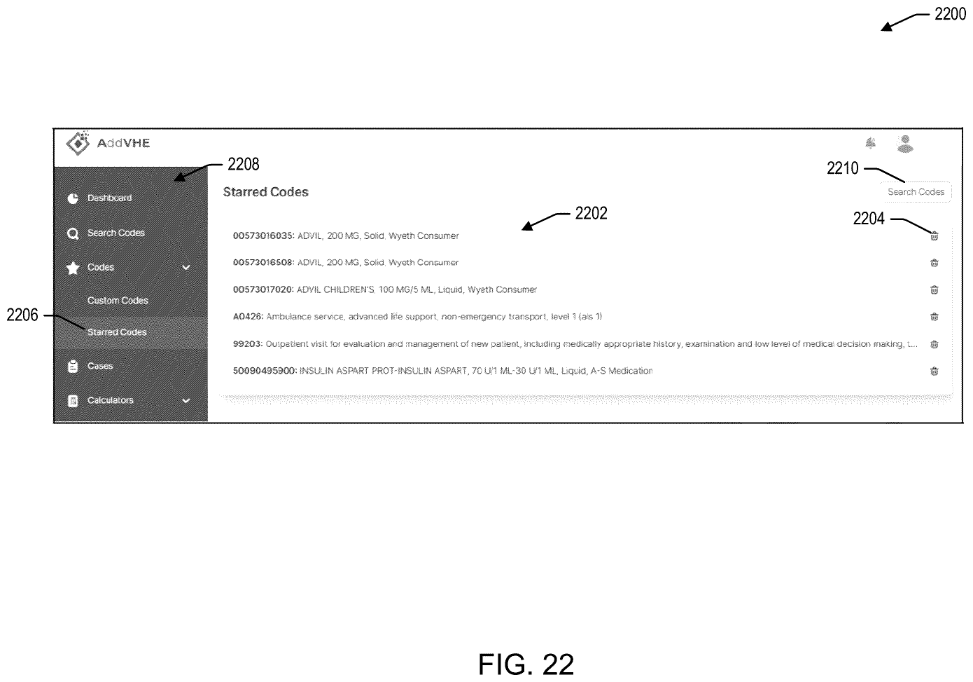

FIG. 22 shows a graphical user interface 2200 displaying a starred code list according to at least one embodiment. Window 2202 can display codes that have been added to a starred code list. The codes can be deleted from the starred codes list by selecting graphic 2204. Graphical user interface 2200 can be accessed by selecting graphic 2206 in menu 2208. In addition, codes can be added to the starred codes list using graphical user interface 2300 which can be accessed by selecting graphic 2210.

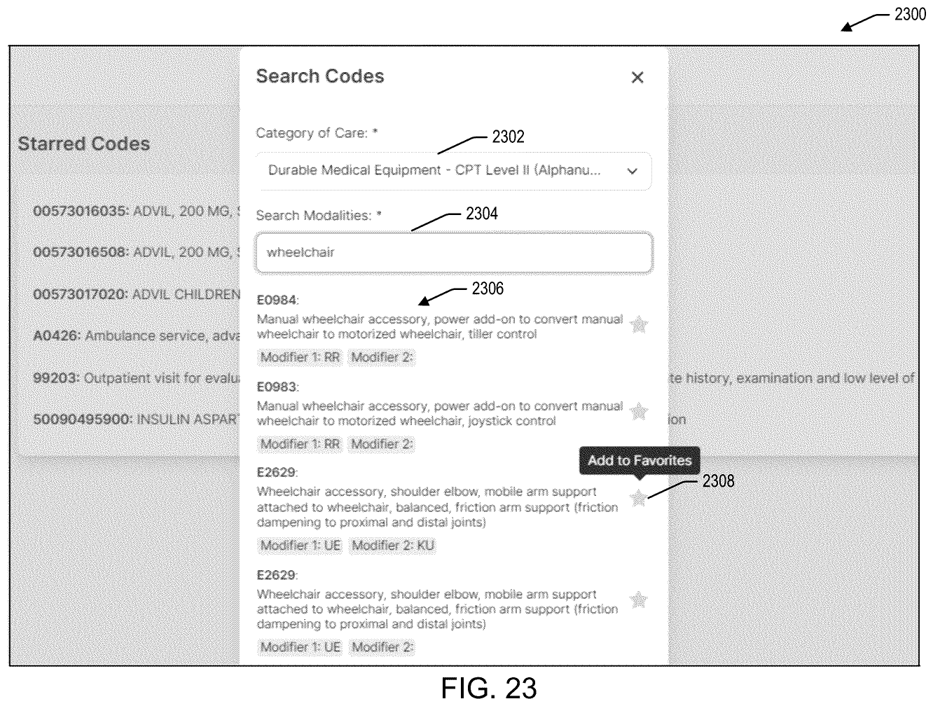

FIG. 23 shows a graphical user interface 2300 for adding codes to a starred code list according to at least one embodiment. Graphical user interface 2300 can be used to search for codes and these codes can be added to the starred code list shown in graphical user interface 2200. A category of remediation for the search can be selected using field 2302, and a search modality can be provided in field 2304. The search modality can be to identify remediation preferences (e.g., codes). The search modalities can include information identifying a remediation preference such as medical codes, keyword, plain text searches or any other search modality. The codes (e.g., remediation preferences) that are returned in response to the search performed using field 2302 and 2304 can be shown in window 2306. One or more of the displayed codes can be added to the starred code list by selecting graphic 2308.

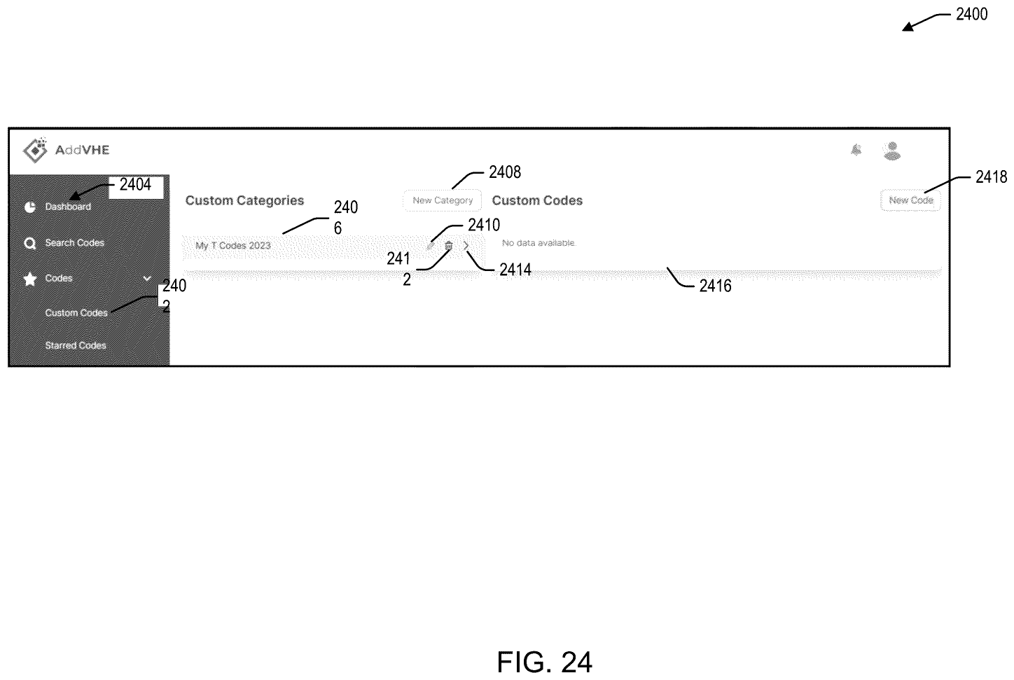

FIG. 24 shows a graphical user interface 2400 for creating custom codes according to at least one embodiment. Custom codes allow a user to incorporate personalized code modalities into a case. Graphical user interface 2400 can be displayed by selecting graphic 2402 in menu 2404. Field 2406 displays a custom category for custom codes. These custom categories can be used to organize custom codes, and, for example, the categories could include custom codes for particular cases, for particular categories of care (e.g., remedial substances, services, equipment, etc.), or for any other organizational paradigm. A new category of remediation can be created by selecting graphic 2408.

Custom categories can be edited, deleted, or expanded so that the codes within the category can be viewed. Selecting graphic 2410 can be selected to edit an existing custom category and graphic 2412 can be selected to delete an existing category of remediation. Graphic 2414 can be selected to expand the custom category shown in field 2406. Expanding a custom category can mean that the codes categorized within the custom category are shown in field 2416. After expanding a custom category, a new code can be added to that category by selecting 2418. Selecting graphic 2418 can cause graphical user interface 2500 to be displayed.

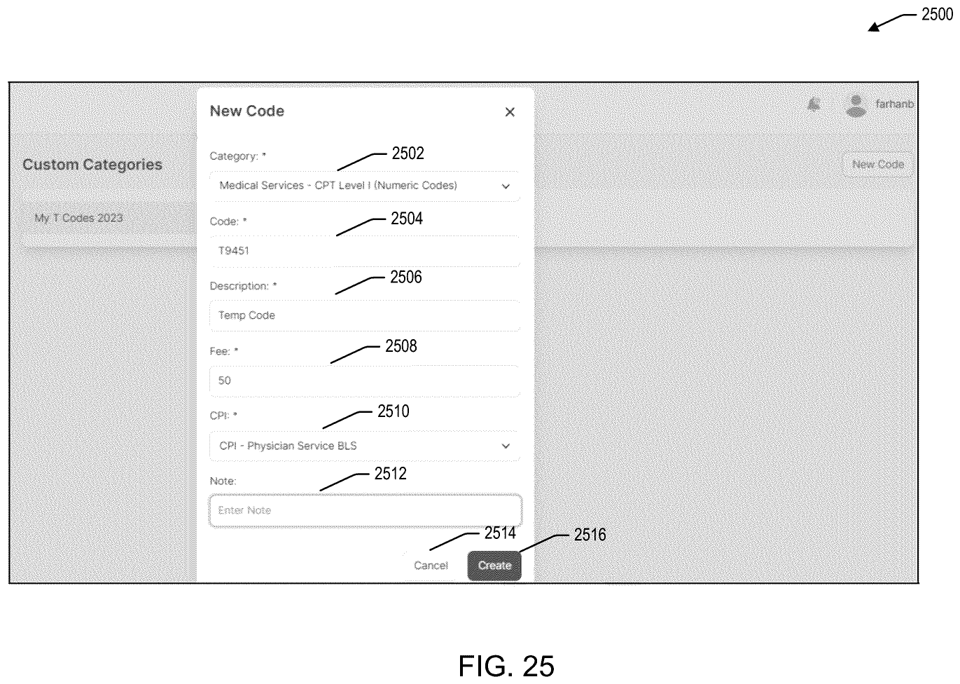

FIG. 25 shows a graphical user interface 2500 for creating a new code according to at least one embodiment. Field 2502 allows a user to select a category for the new code. The category is a category of remediation and the category distinct from the custom category that is used to organize custom codes in graphical user interface 2400. The categories that can be selected using field 2502 can include current procedural terminology (CPT) level I numeric medical service codes, CPT level II alphanumeric medical service codes, CPT level II equipment codes, drug codes, miscellaneous codes, or any other category of medical codes.

The fields shown in graphical user interface 2500 can change based on the category selected in field 2502. For example, a remedial substance can include fields for the national drug code (NDC), the manufacturer name, the product name, the active numerator strength, the ingredients, the route of administration, the package quantity code, the package size, the unit price, whether the remedial substance is a solid or liquid, the consumer price index category, or any other applicable information corresponding to the remedial substance.

A name for the new code can be provided in field 2504, and a description for the new code can be provided in field 2506. A fee corresponding to the code can be provided in field 2508, and a consumer price index (CPI) category can be provided in field 2510. In addition, notes corresponding to the new code can be provided in field 2512. Field 2514 can be selected to discard the new code or field 2516 can be selected to create the new code.

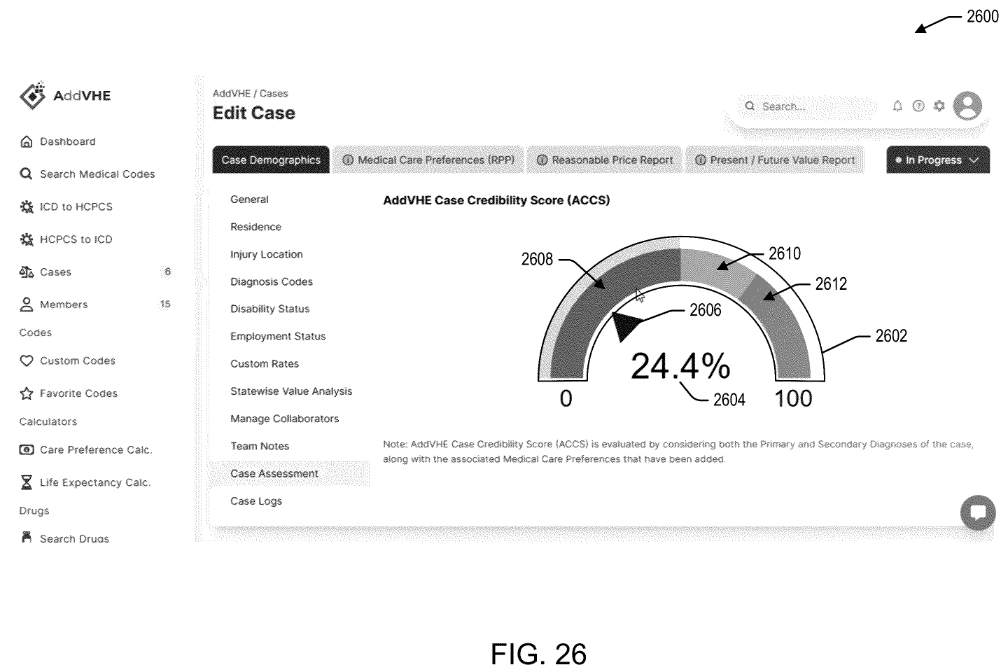

FIG. 26 shows a graphical user interface 2600 for displaying an assessment score according to at least one embodiment. Determining whether a remediation profile includes remediation preferences that are appropriate for a given diagnosis can be challenging because the appropriate remediation preferences can vary between diagnoses. For example, the appropriate codes for a traumatic brain injury can be very different for a cholera diagnosis because there may be little overlap between the treatments for each condition. This challenge can be particularly pronounced for combinations of diagnoses because each unique combination of diagnoses may require a different remediation profile with different, and potentially non-overlapping, care codes. For example, a treatment for a patient with cholera may not be viable for a patient with both cholera and traumatic brain injury.

Visually impaired users may struggle to compare a remediation profile to treatment guidelines because the comparison may require reading and cross referencing the descriptions of individual care codes. The GUI 2600 can enable visually impaired users, or users with physical or mental disabilities that make navigating multiple user interfaces challenging, to receive an indication of whether their remediation profile corresponds to the best practices for their category of remediation (e.g., diagnoses). Users who are the subject of a remediation profile may struggle with a loss of autonomy and they may be reliant on others to complete a remediation profile. This loss of autonomy can mean that a user is not able to evaluate their remediation profile and this can increase the likelihood that the categories of remediation in their remediation profile is not reflective of the user's needs. The graphical user interface 2600, and the other GUIs disclosed herein, can enable a disabled user to evaluate the categories of remediation in their remediation profile without relying on others.

Graphic 2602 can be a visual representation of an evaluation score for a remediation profile. The evaluation score can be determined by the graphical user interface (GUI) engine (e.g., graphical user interface (GUI) engine 230). The engine can access one or more categories of care that correspond to a remediation profile (e.g., from the data store 226 via API endpoint engine 218, or via communication engine 224) and the GUI engine can use the categories of remediation in the remediation profile can be used to access matching remediation preferences (e.g., care guidelines) for the categories of remediation (e.g., from the data store 226 via API endpoint engine 218, or via communication engine 224). The event guidelines can include a list of remedial preferences that correspond to particular categories of remediation in their remediation profile or a particular combination of remediation profiles. The care guidelines (e.g., matching remediation preferences) can be based on medical best practices for a category of remediation (e.g., medical guidelines for a diagnosis).

The evaluation score can be determined by identifying a first number of remediation preferences in the remediation profile, a second number of remediation preferences that are within the remediation profile but not included in the matching remediation preferences, and a third number of remediation preferences that are within both the matching remediation preferences for the categories of remediation in their remediation profile and the remediation preferences within the remediation profile. The evaluation score can be determined by dividing either the second number of remediation preferences or the second number of remediation preferences by the first number of remediation preferences. For example, the graphic 2602 is determined by dividing the third number of remediation preferences by the first number of remediation preferences. This evaluation score can be converted to a percentage, and, for example, the percentage can be displayed as graphic 2604. Graphic 2602 can visually indicate a range of evaluation scores from a minimum possible value (e.g., 0) to a maximum possible value (e.g., 100). Graphic 2606 can indicate a score corresponding to the current remediation profile within graphic 2602. In some embodiments, each care remediation preference can be weighted by the percentage of the total cost of the remediation profile that is represented by that remediation preference.

The graphical element 2602 can be divided into graphics that visually indicate whether the evaluation score exceeds one or more thresholds. For example, graphic 2608 can represent evaluation scores that are below a first evaluation score threshold, graphic 2610 can represent evaluation scores that are above the first evaluation score threshold and below a second evaluation score threshold, and graphic 2612 can represent evaluation scores that are above both the first evaluation score threshold and the second evaluation score threshold. The first and second thresholds can be dynamically updated, and the thresholds based on an evaluation of remediation profiles with the same, or similar, categories of remediation. For example, the first evaluation score threshold can be determined based on the evaluation scores of remediation profiles that were rejected (e.g. by an administrative system) and the second evaluation score threshold can be determined based on the evaluation scores of remediation profiles that were approved with revisions (e.g., by an administrative system).

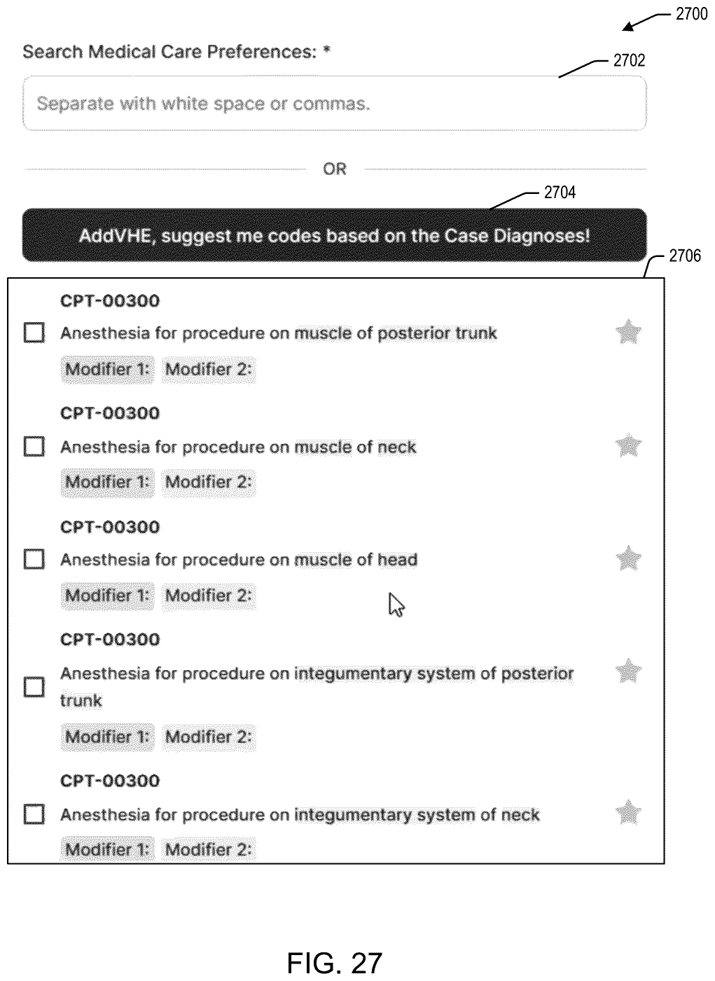

FIG. 27 shows a graphical user interface (GUI) 2700 for contextual remediation preference (e.g., code) suggestions according to at least one embodiment. The graphical user interface 2700 offers an improvement over other interfaces for remediation preference searches (e.g., GUI 2100) because the user interface automatically suggests remediation preferences based on the categories of remediation in a remediation profile. GUI 2700 can enable a search with a single click which can facilitate searches by users with physical or mental disabilities. This automatic suggestion can allow a user to locate appropriate remediation preferences without having to input information into graphical user interface fields (e.g., field 2702). Instead, search results (e.g., remediation preferences) can be returned in response to a single input (e.g., selecting graphic 2704). A single click can cause the graphical user interface engine (e.g., graphical user interface engine 230) to auto populate a query with relevant search information (e.g., information provided in fields 2104-2112), initiate a search request, and to cause a user device (e.g., user device(s) 228) display the remediation preferences returned in response to the query. The search results 2706 can be displayed using any of the techniques for displaying search results described herein (e.g., as described in reference to GUI 2100).

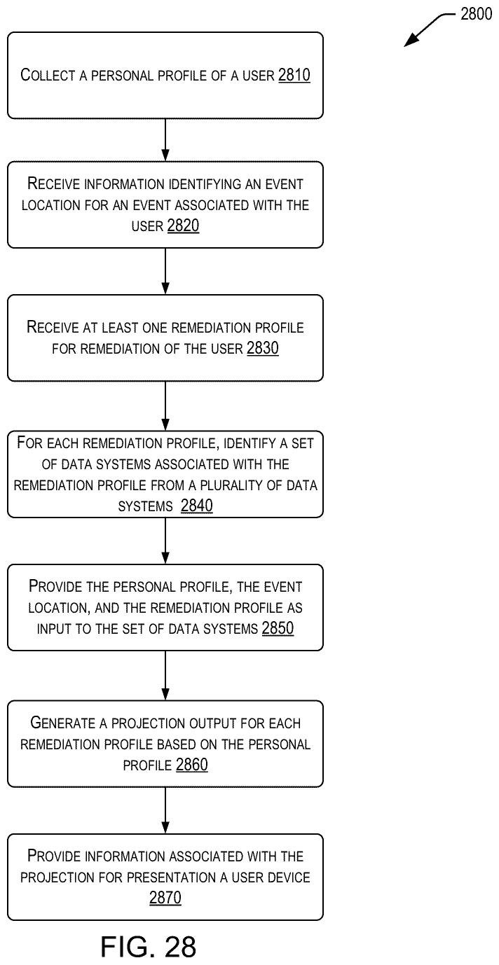

FIG. 28 is a process 2800 for determining a projection according to at least one embodiment. This process is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The orders in which the operations are described are not intended to be construed as a limitation, and any number of the described operations can be combined in any order and/or in parallel to implement the processes or the method.

At block 2810, a personal profile of a user can be collected. The personal profile can be received (e.g., collected) at a computer system (e.g., care system 102, care system 202) from a user device (e.g., user device 104, user device(s) 228) and the personal profile can be provided via a graphical user interface (e.g., graphical user interface 500, graphical user interface 600, graphical user interface 700, graphical user interface 800, graphical user interface 900, graphical user interface 1000, graphical user interface 1100, graphical user interface 1200, graphical user interface 1300, graphical user interface 1400, graphical user interface 1500, graphical user interface 1600, graphical user interface 1700, graphical user interface 1800, graphical user interface 1900, graphical user interface 2000, graphical user interface 2100, graphical user interface 2200, graphical user interface 2300, graphical user interface 2400, graphical user interface 2500, graphical user interface 2600, graphical user interface 2700, etc.). The personal profile can be provided via an application (e.g., a web browser application) executing on a user device such as a mobile phone, a tablet computer, a smartwatch, a laptop computer, and/or any other computing device. The personal profile can be retrieved from a data store (e.g., data store) using a unique identifier associated with the user. For instance, the personal profile may be associated with multiple events and the personal profile may be stored so that the information can be reused or modified without a user having to reenter the information. In some implementations, an event may be associated with multiple personal profiles.

At block 2820, information identifying an event location for an event associated with the user is received. An event can be an injury that caused the subject to suffer from a medical condition. In some implementations, the event location can be a location where the user is to receive remediation for the event. The information identifying a location of an event can be received as part of a personal profile or a remediation profile. The information can be received from a user device (e.g., user device 104, user device(s) 228) and the information can be provided via a graphical user interface (e.g., graphical user interface 500, graphical user interface 600, graphical user interface 700, graphical user interface 800, graphical user interface 900, graphical user interface 1000, graphical user interface 1100, graphical user interface 1200, graphical user interface 1300, graphical user interface 1400, graphical user interface 1500, graphical user interface 1600, graphical user interface 1700, graphical user interface 1800, graphical user interface 1900, graphical user interface 2000, graphical user interface 2100, graphical user interface 2200, graphical user interface 2300, graphical user interface 2400, graphical user interface 2500, graphical user interface 2600, graphical user interface 2700, etc.). The information can be retrieved from a data store (e.g., data store) using a unique identifier associated with the user. Receiving the event location can include providing instructions to cause a user device to display an interactive map and receiving information identifying the event location from the user device. The information identifying the event location can be provided to the user device as input to the interactive map.

At block 2830, at least one remediation profile for the remediation of the user can be received. The remediation profile can be received from a user device (e.g., user device 304, user device(s) 228) and the remediation profile can be provided via a graphical user interface (e.g., graphical user interface 500, graphical user interface 600, graphical user interface 700, graphical user interface 800). The remediation profile can be retrieved from a data store (e.g., data store 334) using a unique identifier associated with the user. Each remediation profile can comprise one or more of a category of remediation, a remedial location designation, a remediation preference type (e.g., a medical care preference type), a service length, a number of annual units, an uncovered equivalent, and/or any additional information about a remediation preference.

A remediation profile can be received through a user interface. For example, instructions can be provided that to cause the user device to display a graphical user interface. One or more categories of remediation for the at least one remediation profile can be received from the user interface. These categories of remediation can be received at the user device as input to the user interface. The plurality of data systems can be queried to identify a plurality of remediation preference types that correspond to the one or more categories of remediation (e.g., suggested or matching remediation preference types). providing, by the computer system, instructions to cause the user device to display at least one of the plurality of matching remediation preference types on the graphical user interface. Information identifying one or more remediation preference types of the plurality of matching remediation preference types can be received from the user device. One or more of the plurality of matching remediation preference types can be added to the at least one remediation profile.

At block 2840, a set of data systems associated with the remediation profile can be identified from a plurality of remediation profiles. A set of remediation profiles can be identified for each remediation profile from 2830. The plurality of data systems can include data system 110, data systems 204, projection system 108, and projection system 220. The data systems can include an equivalency system 206, an equipment system 208, a statistics system 210, a remedial substance system 212, and a service system 214.

At block 2850, the personal profile from 2810, the information from 2820, and a remediation profile from 2830 can be provided as input to the set of data systems identified at 2840. The personal profile, information, and remediation profile may be provided as input to an equivalency system 206, an equipment system 208, a statistics system 210, a remedial substance system 212, and a service system 214, and the output from these systems can be provided as input to projection system 108 or projection system 220.

At block 2860, a projection can be generated for each remediation profile. The projection may be provided as output from the data systems 110, data systems 204, projection system 108, or projection system 220.

At block 2870, information associated with the projection can be provided for presentation at a user device. For example, the information associated with the projection can be provided to the user device with instructions that cause the user device to display the information on a display of the user device. The information can be displayed on a graphical user interface such as the graphical user interfaces described with reference to 2820. The user device can be user device 304, or user device(s) 228 or any other computing system in this disclosure. Presenting the information can include generating a visual representation (e.g., a chart, a graph, a plot, an image, etc.) of the information associated with the projection. For example, this presenting can include any combination of determining a remediation assessment score, generating a visual representation of the remediation assessment score, and providing instructions that cause a user device to display the visual representation as the information associated with the projection.

In some implementations, the event location can be updated using a user interface. After an update, the projection can be updated to reflect the new event location. In some embodiments, the system can change the event location to identify a maximum projection value or a minimum projection value (e.g., the cost of the projection), or to generate a visual representation that compares the projection value across different locations. For example, the visual representation can be a map and the administrative regions in the map can be color coded, or otherwise visually distinguished, so that the user can identify a location with a desired projection value.

The event location can be updated using a map. For example, the updated event location can be received as information identifying an event location from a user device. This event location can be received at the user device as input to an interactive map that is displayed by the user device. The projection can be updated based at least in part on the second event location.

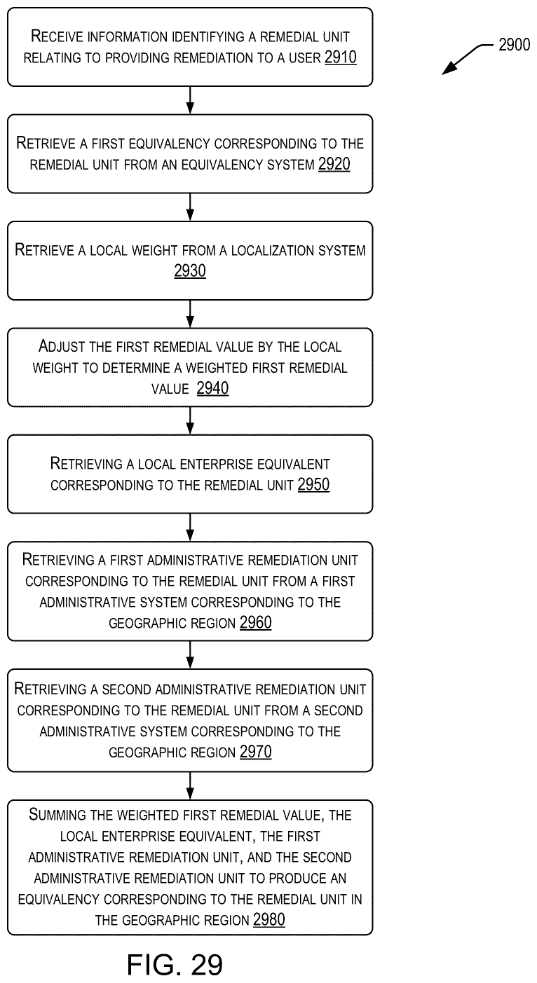

FIG. 29 is a process 2900 for determining an equivalency according to at least one embodiment. This process is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The orders in which the operations are described are not intended to be construed as a limitation, and any number of the described operations can be combined in any order and/or in parallel to implement the processes or the method. Any number of the blocks of any combination of FIGS. 28-34 can be combined in any order to perform the techniques described herein.

At block 2910, information identifying a remedial unit relating to providing remediation to a user can be received. The information can be received by a computer system (e.g., equivalency system 206, equivalency system 322, equivalency projection generator 328) and from a user device (e.g., user device 104, user device(s) 228). The information can be received at the computer system from a mobile device.

At block 2920, a first equivalency corresponding to the remedial unit can be retrieved from a equivalency system 330. The first equivalency can be retrieved by the computer system from block 2910. The first equivalency can be a Medicare cost and the equivalency system can be a Medicare database.

At block 2930, a local weight can be retrieved from a localization system. The localization system can be localization system 332. The local weight can correspond to a administrative region where the user is provided remediation (e.g., where the remedial unit is provided). The administrative region can be a group of states, a state, a region within a state, a city, a neighborhood, a zip code, an address, and/or any other administrative indicator. The local weight can be calculated using a trained machine learning model or calculated using a rules-based approach, or the local weight can be retrieved from a database with precomputed local weights for different administrative regions.

At block 2940, the first equivalency can be adjusted by the first local weight to determine a first weighted equivalency. The adjustment can be performed by the equivalency system 330, the localization system 332, or the computer system from 2910. The first equivalency can be adjusted by multiplying the first equivalency by the first local weight to produce the weighted first equivalency. The weighted first equivalency can be a localized Medicare cost.

At block 2950, a local enterprise equivalent can be retrieved from a enterprise equivalent system. The local enterprise equivalent can correspond to the remedial unit from 2910 and the administrative region from 2920. The local enterprise equivalent can be a markup that is added to the first weighted equivalency from 2940.

At block 2960, a first administrative remediation unit can correspond to the remedial unit can be retrieved. The first administrative remediation unit can be retrieved from a first administrative system 336. The first administrative remediation unit system can correspond to the administrative region from 2920. The first administrative remediation unit can be a Medicaid cost.

At block 2970, a second administrative remediation unit corresponding to the remedial unit can be retrieved. The second administrative remediation unit can be retrieved from a second administrative system 338. The second administrative remediation unit system can correspond to the administrative region from 2920. The second administrative remediation unit can be a Veteran's Administration cost.

At block 2980, the weighted first equivalency, the local enterprise equivalent, the first jurisdictional medical cost, and the second administrative remediation unit can be summed to produce an equivalency corresponding to providing the remedial unit in the administrative region from 2920.

Producing the equivalency can include receiving information from the user, and this information can be provided via a user device. The information may include one or more of an uncovered equivalent, a start date for the item or service corresponding to the equivalency, an end date for the item or service, a minimum cost for the item or service, a maximum cost for the item or service, an average cost for the item or service. The equivalency can be a reasonable value per annum per unit that reflects the annual cost of an item or service. In some embodiments, the equivalency can be a reasonable value over lifetime per unit representing the cost of the item/or service over the life of the subject. The equivalency per annum can be multiplied by the subject's life expectancy or the number of service years for the item or service.

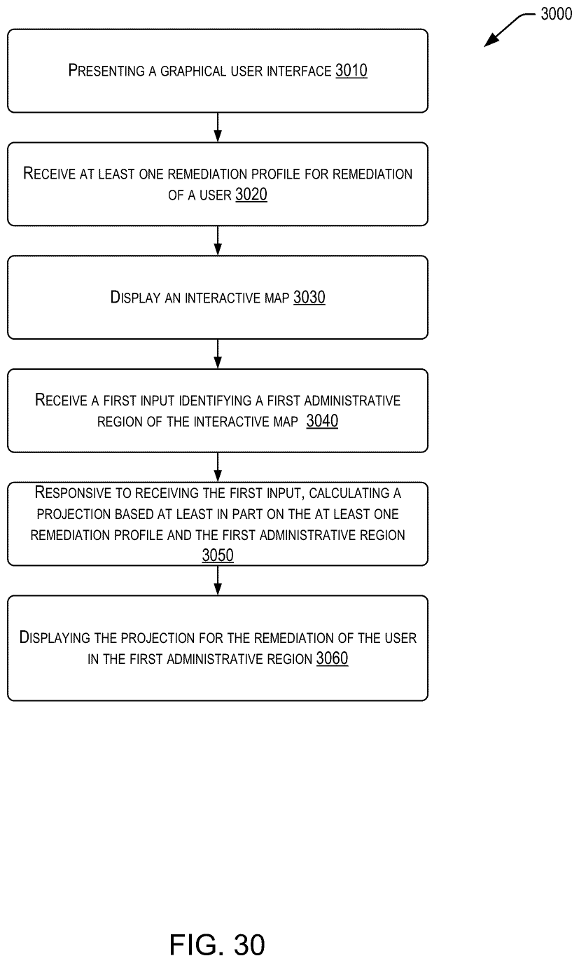

FIG. 30 is a process 3000 for determining projection using an interactive map according to at least one embodiment. This process is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The orders in which the operations are described are not intended to be construed as a limitation, and any number of the described operations can be combined in any order and/or in parallel to implement the processes or the method. Any number of the blocks of any combination of FIGS. 28-34 can be combined in any order to perform the techniques described herein.

At block 3010, a graphical user interface can be presented by a computer system. The graphical user interface can be graphical user interface 500, graphical user interface 600, graphical user interface 700, graphical user interface 800, graphical user interface 900, graphical user interface 1000, graphical user interface 1100, graphical user interface 1200, graphical user interface 1300, graphical user interface 1400, graphical user interface 1500, graphical user interface 1600, graphical user interface 1700, graphical user interface 1800, graphical user interface 1900, graphical user interface 2000, graphical user interface 2100, graphical user interface 2200, graphical user interface 2300, graphical user interface 2400, graphical user interface 2500, graphical user interface 2600, graphical user interface 2700, etc. The computer system can be user device 304, or user device(s) 228. For example, the computer system can be a mobile device such as a mobile phone, a tablet computer, a laptop computer, a smartwatch, and/or any other mobile computing device.

At block 3020, at least one remediation profile can be received. The at least one remediation profile can be received via the graphical user interface from 3010. In some embodiments, multiple graphical user interfaces can be presented. The remediation profile can comprise one or more of a category of remediation, a remedial location designation, a remediation preference type, a service length, a number of annual units, or an uncovered equivalent.

At block 3030, an interactive map can be displayed via a graphical user interface. For example, the graphical user interface can be the graphical user interface 700.

At block 3040, a first input identifying a first administrative region of the interactive map can be received. The first administrative region can be a state, a city, a region of a state, a zip code, or an address. The input identifying the first geographical region can be a selection of a point on a map, or a selection identifying a region of a map. For instance, a point on a map can be provided by a user clicking a region using a cursor and a selection identifying a region of a map could be a shape drawn with the cursor. In some embodiments, the input can be provided by the cursor's current location without further user input (e.g., the cursor “hovering” in a region).

At block 3050, a projection can be generated. The remediation profile can be generated based on the at least one remediation profile and the first administrative region. The projection could be generated for the selection from 3040, and the projection could be updated as other selections are provided. Such an input can allow a user to compare different regions of interest.

At block 3060, the projection can be displayed on the graphical user interface. The projection can be for the medical care of the user in the first administrative region. The projection can be displayed alongside previous projections so that the two projections can be compared.

In some embodiments, a second input identifying a administrative region can be received. In response to receiving the second input, an updated remediation profile can be calculated. This updated remediation profile can be displayed on the graphical user interface.

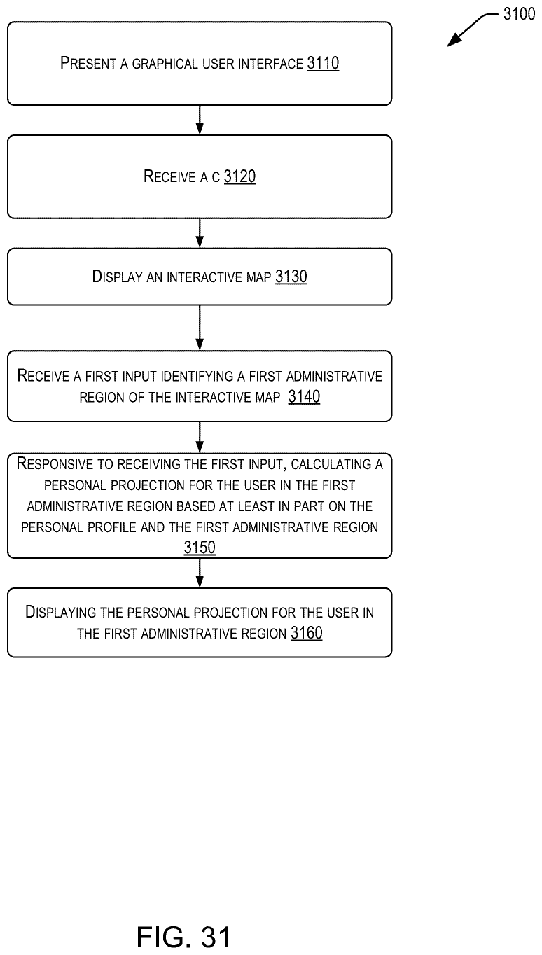

FIG. 31 is a process 3100 for determining a personal projection using an interactive map according to at least one embodiment. This process is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The orders in which the operations are described are not intended to be construed as a limitation, and any number of the described operations can be combined in any order and/or in parallel to implement the processes or the method. Any number of the blocks of any combination of FIGS. 28-34 can be combined in any order to perform the techniques described herein.

At block 3110, a graphical user interface can be presented by a computer system. The graphical user interface can be graphical user interface 500, graphical user interface 600, graphical user interface 700, graphical user interface 800, graphical user interface 900, graphical user interface 1000, graphical user interface 1100, graphical user interface 1200, graphical user interface 1300, graphical user interface 1400, graphical user interface 1500, graphical user interface 1600, graphical user interface 1700, graphical user interface 1800, graphical user interface 1900, graphical user interface 2000, graphical user interface 2100, graphical user interface 2200, graphical user interface 2300, graphical user interface 2400, graphical user interface 2500, graphical user interface 2600, graphical user interface 2700, etc. The computer system can be user device 104, or user device(s) 228. The computer system can be a mobile device such as a mobile phone, a tablet computer, a smartwatch, a laptop computer, and/or any other suitable computing device.

At block 3120, at least one personal profile can be received. The at least one personal profile can be received via the graphical user interface from 3110. In some embodiments, multiple graphical user interfaces can be presented. The personal profile can comprise one or more of a state, a city, a zip code, an address, a birth date for the subject, an ethnicity for the subject, a sex of the subject, or a marital status of the subject.

At block 3130, an interactive map can be displayed via a graphical user interface.

At block 3140, a first input identifying a first administrative region of the interactive map can be received. The first administrative region can be a state, a city, a region of a state, a zip code, or an address. The input identifying the first geographical region can be a selection of a point on a map, or a selection identifying a region of a map. For instance, a point on a map can be provided by a user clicking a region using a cursor and a selection identifying a region of a map could be a shape drawn with the cursor. In some embodiments, the input can be provided by the cursor's current location without further user input (e.g., the cursor “hovering” in a region).

At block 3150, a personal projection (e.g., a demographic projection) can be generated. The personal profile can be generated based on the at least one personal profile and the first administrative region. The personal projection can be a life expectancy for the subject that was used to generate the personal profile in 3120.

At block 3160, the personal projection can be displayed on the graphical user interface. The personal projection can be a life expectancy calculation for the subject that was used to generate the personal profile in 3120. The personal projection can include the information from the personal profile in 3120 in addition to the life expectancy. The personal projection can be used as input to calculate an equivalency or a projection for the subject from 3120.

In some embodiments, a second input identifying an administrative region can be received. In response to receiving the second input, an updated personal profile can be calculated. This updated personal profile can be displayed on the graphical user interface.

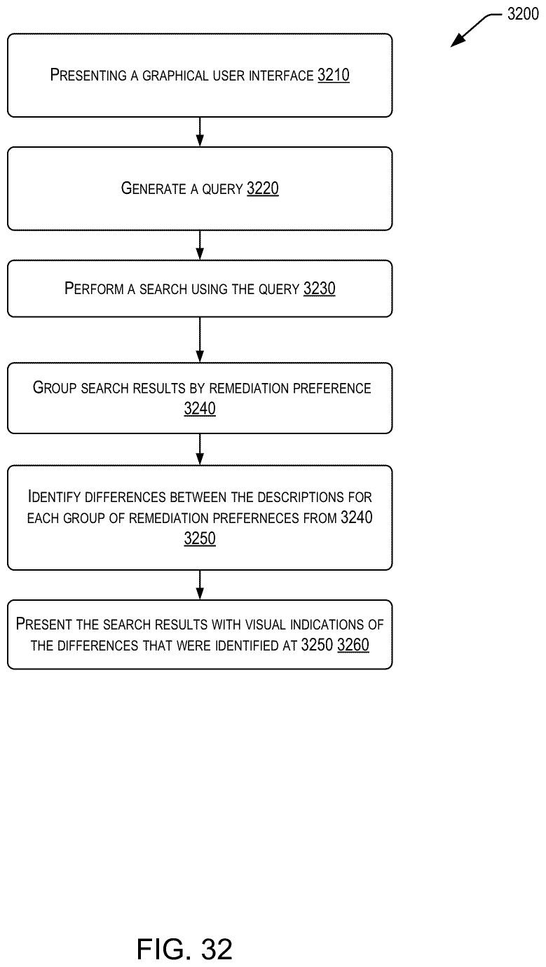

FIG. 32 is a process 3200 for performing a remediation preference search according to at least one embodiment. This process is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The orders in which the operations are described are not intended to be construed as a limitation, and any number of the described operations can be combined in any order and/or in parallel to implement the processes or the method. Any number of the blocks of any combination of FIGS. 28-34 can be combined in any order to perform the techniques described herein.

At block 3210, a graphical user interface can be presented. The graphical user interface can be presented on a user device (e.g., user device(s) 228) in response to instructions received from the care system 202. The graphical user interface can be graphical user interface 500, graphical user interface 600, graphical user interface 700, graphical user interface 800, graphical user interface 900, graphical user interface 1000, graphical user interface 1100, graphical user interface 1200, graphical user interface 1300, graphical user interface 1400, graphical user interface 1500, graphical user interface 1600, graphical user interface 1700, graphical user interface 1800, graphical user interface 1900, graphical user interface 2000, graphical user interface 2100, graphical user interface 2200, graphical user interface 2300, graphical user interface 2400, graphical user interface 2500, graphical user interface 2600, graphical user interface 2700, etc.

At block 3220, a query can be generated. The query can be generated by a graphical user interface engine (e.g., GUI engine 230) of the care system 202 using information received via the graphical user interface presented at 3210. The query can include information corresponding to one or more remediation profiles and/or an event location. The information corresponding to the remediation profile can include any combination of a category of remediation, a remedial location designation, a remediation preference type, a service length, a number of annual units, or an uncovered equivalent.

At block 3230, a search can be performed using the query from 3220. The search can identify results that match the query in any combination of the care system's data stores (e.g., data store 226) or the data systems (e.g., data systems 204). The search can identify one or more remediation preferences as search results.

At block 3240, the search results can be grouped by remediation preference. For example, each remediation preference can be associated with a code, and multiple remediation preference versions can correspond to a single code. Each remedial preference version can be associated with a description, and the remediation preference versions can be arranged sequentially in the graphical user interface.

At block 3250, differences between the descriptions for each group of remediation preferences from 3240 can be determined. The words or characters in the description can compared by the graphical user interface engine. Any discrepancy between the characters or words can be determined to be a difference.

At block 3260, the search results can be presented with visual indications of the differences that were identified at 3250. For example, presenting the search results can include providing instructions to a user device. The instructions can cause the user device to display the search results with a visual indication of the differences identified at 3250. For example, the differences can be underlined, highlighted, enclosed in a box, italicized, changed to a distinctive color, bolded, or otherwise visually indicated.

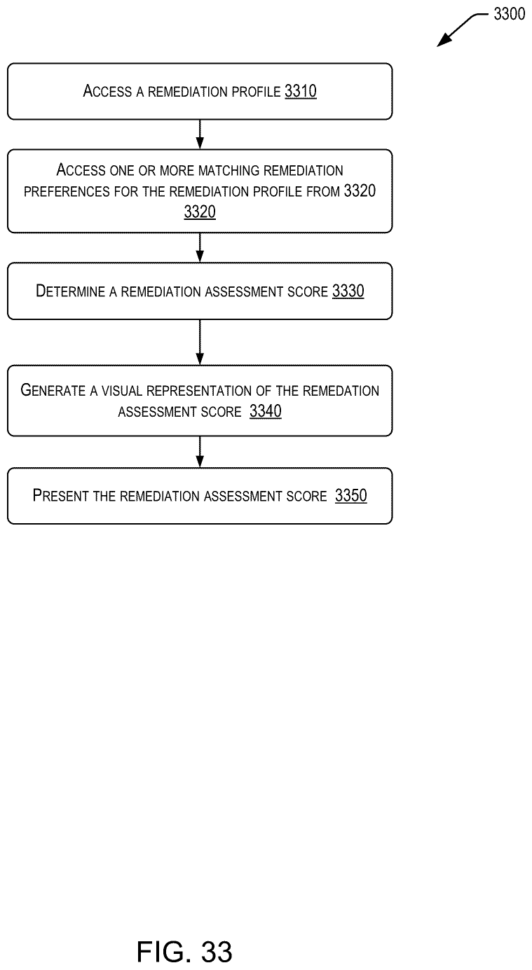

FIG. 33 is a process 3300 for displaying a visual representation of a remediation assessment score according to at least one embodiment. This process is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The orders in which the operations are described are not intended to be construed as a limitation, and any number of the described operations can be combined in any order and/or in parallel to implement the processes or the method. Any number of the blocks of any combination of FIGS. 28-34 can be combined in any order to perform the techniques described herein.

At block 3310, a remediation profile can be accessed. The remediation profile can be accessed by the graphical user interface engine 230 from the data store 226, the data systems 204, or from the user devices 228. The remediation profile can include one or more categories of remediation. The categories of remediation can be a diagnosis of a medical condition in some embodiments.

At block 3320, one or more matching remediation preferences can be accessed. The one or more matching remediation preferences can be for the remediation profile accessed at 3310. The remediation preferences can be accessed by the graphical user interface engine 230 from the data store 226, the data systems 204, or from the user devices 228. The matching remediation preferences can be treatments that correspond to the categories of remediation associated with the remediation profile from 3310. For example, the matching remediation preferences can be recommended care guidelines for a particular diagnosis. The matching remediation preferences can be based on academic literature, treatment guidance from professional organizations, or any other suggested course of treatment for a particular category of remediation.

At block 3330, a remediation assessment score can be determined. The remediation assessment score (e.g., thee evaluation score) can be determined by identifying a first number of remediation preferences in the (e.g., associated with the) remediation profile, a second number of remediation preferences that are within the remediation profile but not included in the matching remediation preferences, and a third number of remediation preferences that are within both the matching remediation preferences for the categories of remediation in their remediation profile and the remediation preferences within the remediation profile. The evaluation score can be determined by dividing either the second number of remediation preferences or the second number of remediation preferences by the first number of remediation preferences.

At block 3340, a visual representation of the remediation assessment score can be generated. For example, the visual representation can be any combination of the graphics shown in graphical user interface 2600.

At block 3350, the remediation assessment score can be presented. The remediation assessment score can be presented on a graphical user interface. The graphical user interface can be presented on a user device (e.g., user device(s) 228) in response to instructions received from the care system 202. The graphical user interface can be graphical user interface 500, graphical user interface 600, graphical user interface 700, graphical user interface 800, graphical user interface 900, graphical user interface 1000, graphical user interface 1100, graphical user interface 1200, graphical user interface 1300, graphical user interface 1400, graphical user interface 1500, graphical user interface 1600, graphical user interface 1700, graphical user interface 1800, graphical user interface 1900, graphical user interface 2000, graphical user interface 2100, graphical user interface 2200, graphical user interface 2300, graphical user interface 2400, graphical user interface 2500, graphical user interface 2600, graphical user interface 2700, etc. Presenting the remediation assessment score or the graphical user interface can include providing instruction to a user device. The instructions can cause the user device to display the visual representation or the graphical user interface.

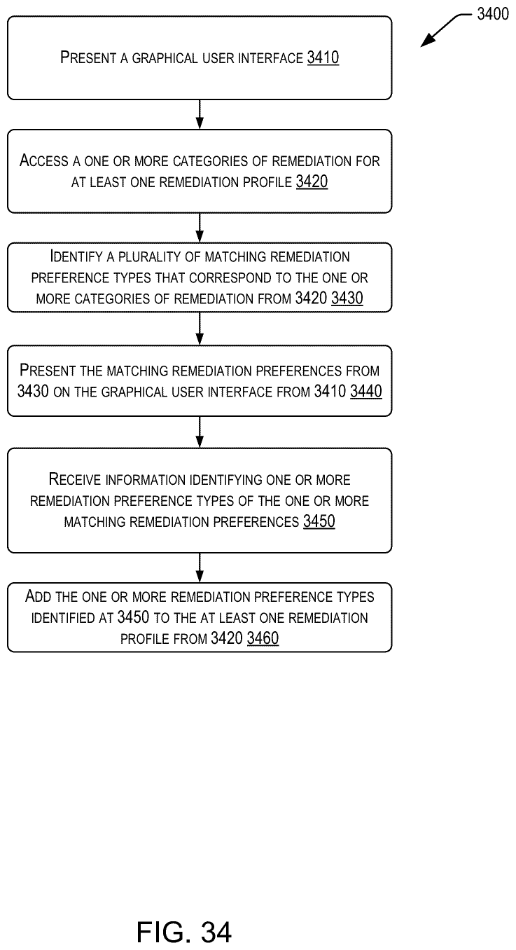

FIG. 34 is a process 3400 for suggesting remediation preferences according to at least one embodiment. This process is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The orders in which the operations are described are not intended to be construed as a limitation, and any number of the described operations can be combined in any order and/or in parallel to implement the processes or the method. Any number of the blocks of any combination of FIGS. 28-34 can be combined in any order to perform the techniques described herein.

At block 3410, a graphical user interface can be presented. The graphical user interface can be graphical user interface 500, graphical user interface 600, graphical user interface 700, graphical user interface 800, graphical user interface 900, graphical user interface 1000, graphical user interface 1100, graphical user interface 1200, graphical user interface 1300, graphical user interface 1400, graphical user interface 1500, graphical user interface 1600, graphical user interface 1700, graphical user interface 1800, graphical user interface 1900, graphical user interface 2000, graphical user interface 2100, graphical user interface 2200, graphical user interface 2300, graphical user interface 2400, graphical user interface 2500, graphical user interface 2600, graphical user interface 2700, etc. Presenting the graphical user interface can include providing instruction to a user device. The instructions can cause the user device to display the graphical user interface.

At block 3420, one or more categories of remediation can be accessed for at least one remediation profile. The categories of remediation can be a diagnosis of a condition that corresponds to a user. The categories of remediation can be intended to treat a user in response to an event. Accessing the one or more categories of remediation can include receiving the one or more categories of remediation from a data store or from a user device (e.g., via a user interface.

At block 3430, a plurality of matching remediation preference types can be identified. The matching remediation preference types can correspond to the one or more categories of remediation from 3420. The matching remediation preference types can be accessed by the graphical user interface engine 230 from the data store 226, the data systems 204, or from the user devices 228. The matching remediation preference types can be treatments that are recommended by academic literature, professional organizations, governmental agencies, and/or insurance agencies.

At block 3440, the matching remediation preferences from 3430 can be presented on the graphical user interface from 3410. Presenting the graphical user interface can include providing instruction to a user device. The instructions can cause the user device to display the graphical user interface.

At block 3450, information identifying one or more remediation preference types can be received. The information can be received via the graphical user interface from 3410.

At block 3460, the one or more remediation preference types identified at 3420 can be added to the at least one remediation profile from 3420. The one or more remediation preference types can be added by the projection system 220 and/or the graphical user interface engine 230.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

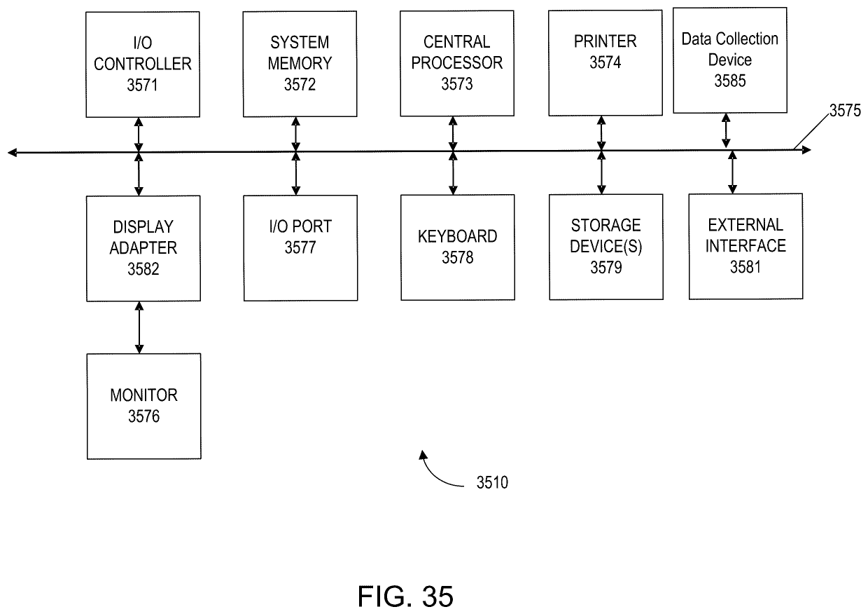

Any of the computer systems mentioned herein may utilize any suitable number of subsystems. Examples of such subsystems are shown in FIG. 35 in computer system 3510. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 35 are interconnected via a system bus 3575. Additional subsystems such as a printer 3574, keyboard 3578, storage device(s) 3579, monitor 3576 (e.g., a display screen, such as an LED), which is coupled to display adapter 3582, and others are shown. Peripherals and input/output (I/O) devices, which couple to I/O controller 3571, can be connected to the computer system by any number of means known in the art such as input/output (I/O) port 3577 (e.g., USB, FireWire®). For example, I/O port 3577 or external interface 3581 (e.g. Ethernet, Wi-Fi, etc.) can be used to connect computer system 3510 to a wide area network such as the Internet, a mouse input device, or a scanner. The interconnection via system bus 3575 allows the central processor 3573 to communicate with each subsystem and to control the execution of a plurality of instructions from system memory 3572 or the storage device(s) 3579 (e.g., a fixed disk, such as a hard drive, or optical disk), as well as the exchange of information between subsystems. The system memory 3572 and/or the storage device(s) 3579 may embody a computer readable medium. Another subsystem is a data collection device 3585, such as a camera, microphone, accelerometer, and the like. Any of the data mentioned herein can be output from one component to another component and can be output to the user.

A computer system can include a plurality of the same components or subsystems, e.g., connected together by external interface 3581, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

Computer programs typically comprise one or more instructions set at various times in various memory devices of a computing device, which, when read and executed by at least one processor, will cause a computing device to execute functions involving the disclosed techniques. In some embodiments, a carrier containing the aforementioned computer program product is provided. The carrier is one of an electronic signal, an optical signal, a radio signal, or a non-transitory computer-readable storage medium.

Any or all of the features and functions described above can be combined with each other, except to the extent it may be otherwise stated above or to the extent that any such embodiments may be incompatible by virtue of their function or structure, as will be apparent to persons of ordinary skill in the art. Unless contrary to physical possibility, it is envisioned that (i) the methods/steps described herein may be performed in any sequence and/or in any combination, and (ii) the components of respective embodiments may be combined in any manner.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims, and other equivalent features and acts are intended to be within the scope of the claims.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. Furthermore, use of “e.g.,” is to be interpreted as providing a non-limiting example and does not imply that two things are identical or necessarily equate to each other.

Unless the context clearly requires otherwise, throughout the description and the claims, the words “comprise,” “comprising,” and the like are to be construed in an inclusive sense, as opposed to an exclusive or exhaustive sense, i.e., in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof.

Additionally, the words “herein,” “above,” “below,” and words of similar import, when used in this application, refer to this application as a whole and not to any particular portions of this application. Where the context permits, words using the singular or plural number may also include the plural or singular number respectively. The word “or” in reference to a list of two or more items, covers all of the following interpretations of the word: any one of the items in the list, all of the items in the list, and any combination of the items in the list. Likewise the term “and/or” in reference to a list of two or more items, covers all of the following interpretations of the word: any one of the items in the list, all of the items in the list, and any combination of the items in the list.

Conjunctive language such as the phrase “at least one of X, Y and Z,” unless specifically stated otherwise, is understood with the context as used in general to convey that an item, term, etc. may be either X, Y or Z, or any combination thereof. Thus, such conjunctive language is not generally intended to imply that certain embodiments require at least one of X, at least one of Y and at least one of Z to each be present. Further, use of the phrase “at least one of X, Y or Z” as used in general is to convey that an item, term, etc. may be either X, Y or Z, or any combination thereof.

In some embodiments, certain operations, acts, events, or functions of any of the algorithms described herein can be performed in a different sequence, can be added, merged, or left out altogether (e.g., not all are necessary for the practice of the algorithms). In certain embodiments, operations, acts, functions, or events can be performed concurrently, e.g., through multi-threaded processing, interrupt processing, or multiple processors or processor cores or on other parallel architectures, rather than sequentially.

Systems and modules described herein may comprise software, firmware, hardware, or any combination(s) of software, firmware, or hardware suitable for the purposes described. Software and other modules may reside and execute on servers, workstations, personal computers, computerized tablets, PDAs, and other computing devices suitable for the purposes described herein. Software and other modules may be accessible via local computer memory, via a network, via a browser, or via other means suitable for the purposes described herein. Data structures described herein may comprise computer files, variables, programming arrays, programming structures, or any electronic information storage schemes or methods, or any combinations thereof, suitable for the purposes described herein. User interface elements described herein may comprise elements from graphical user interfaces, interactive voice response, command line interfaces, and other suitable interfaces.

Further, processing of the various components of the illustrated systems can be distributed across multiple machines, networks, and other computing resources. Two or more components of a system can be combined into fewer components. Various components of the illustrated systems can be implemented in one or more virtual machines or an isolated execution environment, rather than in dedicated computer hardware systems and/or computing devices. Likewise, the data repositories shown can represent physical and/or logical data storage, including, e.g., storage area networks or other distributed storage systems. Moreover, in some embodiments the connections between the components shown represent possible paths of data flow, rather than actual connections between hardware. While some examples of possible connections are shown, any of the subset of the components shown can communicate with any other subset of components in various implementations.

Embodiments are also described above with reference to flow chart illustrations and/or block diagrams of methods, apparatus (systems) and computer program products. Each block of the flow chart illustrations and/or block diagrams, and combinations of blocks in the flow chart illustrations and/or block diagrams, may be implemented by computer program instructions. Such instructions may be provided to a processor of a general purpose computer, special purpose computer, specially-equipped computer (e.g., comprising a high-performance database server, a graphics subsystem, etc.) or other programmable data processing apparatus to produce a machine, such that the instructions, which execute via the processor(s) of the computer or other programmable data processing apparatus, create means for implementing the acts specified in the flow chart and/or block diagram block or blocks. These computer program instructions may also be stored in a non-transitory computer-readable memory that can direct a computer or other programmable data processing apparatus to operate in a particular manner, such that the instructions stored in the computer-readable memory produce an article of manufacture including instruction means which implement the acts specified in the flow chart and/or block diagram block or blocks. The computer program instructions may also be loaded to a computing device or other programmable data processing apparatus to cause operations to be performed on the computing device or other programmable apparatus to produce a computer implemented process such that the instructions which execute on the computing device or other programmable apparatus provide steps for implementing the acts specified in the flow chart and/or block diagram block or blocks.

Any patents and applications and other references noted above, including any that may be listed in accompanying filing papers, are incorporated herein by reference. Aspects of the invention can be modified, if necessary, to employ the systems, functions, and concepts of the various references described above to provide yet further implementations of the invention. These and other changes can be made to the invention in light of the above Detailed Description. While the above description describes certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

To reduce the number of claims, certain aspects of the invention are presented below in certain claim forms, but the applicant contemplates other aspects of the invention in any number of claim forms. For example, while only one aspect of the invention is recited as a means-plus-function claim under 35 U.S.C sec. 112(f) (AIA), other aspects may likewise be embodied as a means-plus-function claim, or in other forms, such as being embodied in a computer-readable medium. Any claims intended to be treated under 35 U.S.C. § 112(f) will begin with the words “means for,” but use of the term “for” in any other context is not intended to invoke treatment under 35 U.S.C. § 112(f). Accordingly, the applicant reserves the right to pursue additional claims after filing this application, in either this application or in a continuing application.

What is claimed is:

1. A computer-implemented method comprising:
collecting, by a computer system, a personal profile of a user;
receiving, by the computer system, information identifying a first event location for an event associated with the user;
receiving, by the computer system, at least one remediation profile for remediation of the user, wherein each remediation profile comprises one or more of a category of remediation, a remedial location designation, a remediation preference type, a service length, a number of annual units, or an uncovered equivalent;
for each remediation profile:
identifying, from a plurality of data systems, a set of data systems associated with the remediation profile, each data system comprising one or more of an equivalency system, an equipment system, a statistics system, a remedial substance system, or a service system;
providing the personal profile, the first event location, and the remediation profile as input to the set of data systems;
generating, using the set of data systems, a projection as output for each remediation profile; and
providing, for presentation at a user device, information associated with the projection for each remediation profile.

2. The method of claim 1, wherein providing information associated with the projection for each remediation profile comprises, for each remediation profile:
determining, by the computer system, a remediation assessment score;
generating, by the computer system, a visual representation of the remediation assessment score; and
providing, by the computer system, instructions that cause a user device to display the visual representation as the information associated with the projection.

3. The method of claim 1, wherein receiving information identifying the first event location comprises:
providing, by the computer system, instructions to cause the user device to display a graphical user interface comprising an interactive map; and
receiving, by the computer system, information identifying a first event location from the user device, wherein the information identifying the first event location is provided as input to the interactive map.

4. The method of claim 3 wherein providing the information associated with the projection for each remediation profile comprises:
receiving, by the computer system, information identifying a second event location from the user device, wherein the information identifying the second event location is provided as input to the interactive map; and
updating, by the computer system, the projection based at least in part on the second event location.

5. The method of claim 1, wherein receiving the at least one remediation profile comprises:
providing, by the computer system, instructions to cause the user device to display a graphical user interface;
receiving, by the computer system and from the user device, one or more categories of remediation for the at least one remediation profile;
querying, by the computer system, the plurality of data systems to identify a plurality of matching remediation preference types that correspond to the one or more categories of remediation; and
providing, by the computer system, instructions to cause the user device to display at least one of the plurality of matching remediation preference types on the graphical user interface.

6. The method of claim 5, further comprising:
receiving, by the computer system, information identifying one or more remediation preference types of the plurality of matching remediation preference types; and
adding, by the computer system, the one or more of the plurality of matching remediation preference types to the at least one remediation profile.

7. The method of claim 1, wherein the event location is a location at which the remediation of the user is to be performed.

8. A computer system, comprising:
one or more memories; and
one or more processors in communication with the one or more memories and configured to execute instructions stored in the one or more memories to performing a process comprising:
collecting a personal profile of a user;
receiving information identifying a first event location for an event associated with the user;
receiving at least one remediation profile for remediation of the user, wherein each remediation profile comprises one or more of a category of remediation, a remedial location designation, a remediation preference type, a service length, a number of annual units, or an uncovered equivalent;
for each remediation profile:
identifying, from a plurality of data systems, a set of data systems associated with the remediation profile, each data system comprising one or more of an equivalency system, an equipment system, a statistics system, a remedial substance system, or a service system;
providing the personal profile, the first event location, and the remediation profile as input to the set of data systems;
generating, using the set of data systems, a projection as output for each remediation profile; and
providing, for presentation at a user device, information associated with the projection for each remediation profile.

9. The computer system of claim 8, wherein providing information associated with the projection for each remediation profile comprises, for each remediation profile:
determining a remediation assessment score;
generating a visual representation of the remediation assessment score; and
providing instructions that cause a user device to display the visual representation as the information associated with the projection.

10. The computer system of claim 8, wherein receiving information identifying the first event location comprises:
providing instructions to cause the user device to display a graphical user interface comprising an interactive map; and
receiving information identifying a first event location from the user device, wherein the information identifying the first event location is provided as input to the interactive map.

11. The computer system of claim 10, wherein providing the information associated with the projection for each remediation profile comprises:
receiving information identifying a second event location from the user device, wherein the information identifying the second event location is provided as input to the interactive map; and
updating the projection based at least in part on the second event location.

12. The computer system of claim 8, wherein receiving the at least one remediation profile comprises:
providing instructions to cause the user device to display a graphical user interface;
receiving, from the user device, one or more categories of remediation for the at least one remediation profile;
querying the plurality of data systems to identify a plurality of matching remediation preference types that correspond to the one or more categories of remediation; and
providing instructions to cause the user device to display at least one of the plurality of matching remediation preference types on the graphical user interface.

13. The computer system of claim 12, further comprising:
receiving information identifying one or more remediation preference types of the plurality of matching remediation preference types; and
adding the one or more of the plurality of matching remediation preference types to the at least one remediation profile.

14. The computer system of claim 8, wherein the event location is a location at which the remediation of the user is to be performed.

15. A non-transitory computer-readable medium having stored thereon instructions that, when executed by one or more processors, cause the one or more processors to perform a process comprising:
collecting a personal profile of a user;
receiving information identifying a first event location for an event associated with the user;
receiving at least one remediation profile for remediation of the user, wherein each remediation profile comprises one or more of a category of remediation, a remedial location designation, a remediation preference type, a service length, a number of annual units, or an uncovered equivalent;
for each remediation profile:
identifying, from a plurality of data systems, a set of data systems associated with the remediation profile, each data system comprising one or more of an equivalency system, an equipment system, a statistics system, a remedial substance system, or a service system;
providing the personal profile, the first event location, and the remediation profile as input to the set of data systems;
generating, using the set of data systems, a projection as output for each remediation profile; and
providing, for presentation at a user device, information associated with the projection for each remediation profile.

16. The non-transitory computer-readable medium of claim 15, wherein providing information associated with the projection for each remediation profile comprises, for each remediation profile:
determining a remediation assessment score;
generating a visual representation of the remediation assessment score; and
providing instructions that cause a user device to display the visual representation as the information associated with the projection.

17. The non-transitory computer-readable medium of claim 15, wherein receiving information identifying the first event location comprises:
providing instructions to cause the user device to display a graphical user interface comprising an interactive map; and
receiving information identifying a first event location from the user device, wherein the information identifying the first event location is provided as input to the interactive map.

18. The non-transitory computer-readable medium of claim 17, wherein providing the information associated with the projection for each remediation profile comprises:
receiving information identifying a second event location from the user device, wherein the information identifying the second event location is provided as input to the interactive map; and
updating the projection based at least in part on the second event location.

19. The non-transitory computer-readable medium of claim 15, wherein receiving the at least one remediation profile comprises:
providing instructions to cause the user device to display a graphical user interface;
receiving, from the user device, one or more categories of remediation for the at least one remediation profile;
querying the plurality of data systems to identify a plurality of matching remediation preference types that correspond to the one or more categories of remediation; and
providing instructions to cause the user device to display at least one of the plurality of matching remediation preference types on the graphical user interface.

20. The non-transitory computer-readable medium of claim 19, further comprising:
receiving information identifying one or more remediation preference types of the plurality of matching remediation preference types; and
adding the one or more of the plurality of matching remediation preference types to the at least one remediation profile.
